SEQUENCE LISTING (2 sequences)

SEQ ID NO: 1 - protein, 569 aa, Homo sapiens
IESLRVTVDFLKVPLGLKKPVLKEVAVGPPKRPQPAALERYKARRSDAMDTESQYSGYSYKSGHSRSSRKHRDRRDRHRSKSRDGGRGDKSVTIQAPGEPLLDNESTRGDERDDNWGETTTVVTGTSEHSISHDDLTRIAKDMEDSVPLDCSRHLGVAAGATLALLSFLTPLAFLLLPPLLWREELEPCGTACEGLFISVAFKLLILLLGSWALFFRRPKASLPRVFVLRALLMVLVFLLVVSYWLFYGVRILDARERSYQGVVQFAVSLVDALLFVHYLAVVLLELRQLQPQFTLKVVRSTDGASRFYNVGHLSIQRVAVWILEKYYHDFPVYNPALLNLPKSVLAKKVSGFKVYSLGEENSTNNSTGQSRAVIAAAARRRDNSHNEYYYEEAEHERRVRKRRARLVVAVEEAFTHIKRLQEEEQKNPREVMDPREAAQAIFASMARAMQKYLRTTKQQPYHTMESILQHLEFCITHDMTPKAFLERYLAAGPTIQYHKERWLAKQWTLVSEEPVTNGLKDGIVFLLKRQDFSLVVSTKKVPFFKLSEEFVDPKSHKFVMRLQSETSV

SEQ ID NO: 2 - protein, 440 aa, Homo sapiens
MASLTVKAYLLGKEDAAREIRRFSFCCSPEPEAEAEAAAGPGPCERLLSRVAALFPALRPGGFQAHYRDEDGDLVAFSSDEELTMAMSYVKDDIFRIYIKEKKECRRDHRPPCAQEAPRNMVHPNVICDGCNGPVVGTRYKCSVCPDYDLCSVCEGKGLHRGHTKLAFPSPFGHLSEGFSHSRWLRKVKHGHFGWPGWEMGPPGNWSPRPPRAGEARPGPTAESASGPSEDPSVNFLKNVGESVAAALSPLGIEVDIDVEHGGKRSRLTPVSPESSSTEEKSSSQPSSCCSDPSKPGGNVEGATQSLAEQMRKIALESEGRPEEQMESDNCSGGDDDWTHLSSKEVDPSTGELQSLQMPESEGPSSLDPSQEGPTGLKEAALYPHLPPEADPRLIESLSQMLSMGFSDEGGWLTRLLQTKNYDIGAALDTIQYSKHPPPL

(19) United States
(12) Patent Application Publication
(10) Pub. No.: US 2016/0032005 A1
(43) Pub. Date: Feb. 4, 2016

(54) METHODS AND PHARMACEUTICALS COMPOSITIONS FOR TREATING BREAST CANCERS

(71) Applicants: INSERM (Institut National de la Sante et de la Recherche Medicale), Paris (FR); UNIVERSITE D'AIX MARSEILLE, Marseille Cedex 07 (FR); INSTITUT JEAN PAOLI & IRENE CALMETTES, Paris Marseille (FR)

(72) Inventors: Jean-Paul BORG, Marseille (FR); Francois BERTUCCI, Marseille (FR); Tania PUVIRAJESINGHE, Marseille (FR)

(21) Appl. No.: 14/779,478

(22) PCT Filed: Mar. 28, 2014
(86) PCT No.: PCT/EP2014/056283

§ 371 Date: Sep. 23, 2015

(51) Int. Cl. C07K 16/30; C12N 15/113; G01N 33/68; C07K 14/82; C12Q 1/68; A61K 47/48; A61K 31/436

(52) U.S. Cl. CPC C07K 16/3015; A61K 47/48584; C12N 15/1138; A61K 31/436; C07K 14/82; C12Q 1/6886; G01N 33/6872; C07K 2317/73; C12N 2310/14; C12N 2320/30; A61K 38/00

(57) ABSTRACT

The present invention relates to methods and pharmaceutical compositions for treating breast cancers. In particular, the present invention relates to a method for predicting the survival of a patient suffering from a breast cancer comprising i) determining the expression level of Vangl2 in a tumor sample obtained from the patient, ii) comparing the expression level determined at step i) with a predetermined reference value and iii) providing a poor prognosis when the expression level determined at step i) is higher than the predetermined reference value. The present invention also relates to a method for treating a patient suffering from a breast cancer comprising the steps consisting of i) predicting the survival of the patient according to claim 1 and ii) administering the patient with an anti-Vangl2 antibody or an inhibitor of Vangl2 expression or an inhibitor of the Vangl2-p62 interaction when it is concluded that the patient has a poor prognosis at step i).

### FIELD OF THE INVENTION:

The present invention relates to methods and pharmaceutical compositions for treating breast cancers.

### BACKGROUND OF THE INVENTION:

Breast cancers are molecularly heterogenous and are classified in five subtypes according to gene expression profiles [28, 29]. For example, basal-like breast cancers, also called triple negative breast cancers (because they lack hormone receptor (ER, PR) and HER2 expression), represent 15% of total breast cancers. Basal-like subtype is termed after the basal epithelial layer cells due to their similarities in gene expression pattern. Basal breast cancer typically expresses basal cytokeratins such as CK5/6, CK17 as well as cadherin, and epidermal growth factor receptor (EGFR) [2]. This type of cancer is very aggressive, has a high rate of metastases, and carries a poor prognosis. Furthermore basal-like breast cancers are resistant to chemotherapy and are a leading cause of mortality. Basal-type breast cancer is also associated with a lack of proven therapy, due to the complexity of this disease and the various subtypes [30]. Despite new approaches that comprise optimization of common cytotoxic agents (addition of platinum salts, dose intensification strategies) and introduction of novel agents (i.e. poly-ADP-ribose-polymerase-1 inhibitors, EGFR and anti-angiogenic inhibitors), there is still a strong need to identify novel therapeutic targets in this chemotherapy-resistant entity. Previous works found expression of Vangl2 in breast cancer cells [27]. Vangl2 is a mammalian four-transmembrane cell surface receptor localized in intracellular and membrane cell compartments. The protein is a cell surface receptor that plays a major role in planar cell polarity and embryonic development. Loss-of-function mutations lead to dramatic neural tube defects in mice and humans. The mode of action of Vangl2 in the tumorigenicity of breast basal-like cancer remains largely unknown.

### SUMMARY OF THE INVENTION:

The present invention relates to methods and pharmaceutical compositions for treating breast cancers. In particular, the present invention relates to a method for predicting the survival of a patient suffering from a breast cancer comprising i) determining the expression level of Vangl2 in a tumor sample obtained from the patient, ii) comparing the expression level determined at step i) with a predetermined reference value and iii) providing a poor prognosis when the expression level determined at step i) is higher than the predetermined reference value. The present invention also relates to a method for treating a patient suffering from a breast cancer comprising the steps consisting of i) predicting the survival of the patient according to claim 1 and ii) administering the patient with an anti-Vangl2 antibody or an inhibitor of Vangl2 expression or an inhibitor of Vangl2 when it is concluded that the patient has a poor prognosis at step i).

### DETAILED DESCRIPTION OF THE INVENTION:

To gain further insights into the functions of Vangl2, the inventors purified Vangl2 at the endogenous level and identified p62/sequestosome-1, a multi-domain adaptor protein, as a novel Vangl2 partner. p62 plays a role in autophagy and is endowed with oncogenic properties. Analysis of human breast cancer samples revealed that Vangl2 is overexpressed in basal-like cancers at the genomic, transcriptomic and protein levels. In addition, in vivo assays in nude mice demonstrate the contribution of Vangl2 to breast cancer development. The inventors found that downregulation of Vangl2 expression as well as disruption of the Vangl2-p62 complex decreased tumorigenicity, cell migration and JNK activation of breast cancer cells. Together these observations establish the role of Vangl2, a cell polarity receptor, in basal-like breast cancer and furthermore suggest that inhibition of Vangl2-p62 interaction may represent a novel therapeutic strategy for the treatment of this disease.

An aspect of the present invention relates to a method for predicting the survival of a patient suffering from a breast cancer comprising i) determining the expression level of Vangl2 in a tumor sample obtained from the patient, ii) comparing the expression level determined at step i) with a predetermined reference value and iii) providing a poor prognosis when the expression level determined at step i) is higher than the predetermined reference value.

In some embodiments, the patient suffers from a basal breast cancer, a metastatic breast cancer or a triple negative breast cancer. As used herein the expression “Triple negative breast cancer” has its general meaning in the art and means that said breast cancer lacks receptors for the hormones estrogen (ER-negative) and progesterone (PR-negative), and for the protein HER2.

As used herein the term “Vangl2” has its general meaning in the art and refers to VANGL planar cell polarity protein 2. An exemplary amino sequence is SEQ ID NO: 1:

(Vangl2_homo sapiens)
SEQ ID NO: 1
IESLRVTVDF LKVPLGLKKP VLKEVAVGPP KRPQPAALER
YKARRSDA MDTESQYSGY SYKSGHSRSS RKHRDRRDRH
RSKSRDGGRG DKSVTIQAPG EPLLDNESTR GDERDDNWGE
TTTVVTGTSE HSISHDDLTR IAKDMEDSVP LDCSRHLGVA
AGATLALLSF LTPLAFLLLP PLLWREELEP CGTACEGLFI
SVAFKLLILL LGSWALFFRR PKASLPRVFV LRALLMVLVF
LLVVSYWLFY GVRILDARER SYQGVVQFAV SLVDALLFVH
YLAVVLLELR QLQPQFTLKV VRSTDGASRF YNVGHLSIQR
VAVWILEKYY HDFPVYNPAL LNLPKSVLAK KVSGFKVYSL
GEENSTNNST GQSRAVIAAA ARRRDNSHNE YYYEEAEHER
RVRKRRARLV VAVEEAFTHI KRLQEEEQKN PREVMDPREA
AQAIFASMAR AMQKYLRTTK QQPYHTMESI LQHLEFCITH
DMTPKAFLER YLAAGPTIQY HKERWLAKQW TLVSEEPVTN
GLKDGIVFLL KRQDFSLVVS TKKVPFFKLS EEFVDPKSHK
FVMRLQSETS V

The term “tumor sample” means any tissue sample derived from the tumor of the patient. The tissue sample is obtained for the purpose of the in vitro evaluation. The sample can be fresh, frozen, fixed (e.g., formalin fixed), or embedded (e.g., paraffin embedded). In a particular embodiment the sample results from biopsy performed in a tumour sample of the patient.

Determining an expression level of a gene in a tumor sample obtained from a patient can be implemented by a panel of techniques well known in the art. Typically, an expression level of a gene is assessed by determining the quantity of mRNA produced by this gene.

Methods for determining a quantity of mRNA are well known in the art. For example nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The thus extracted mRNA is then detected by hybridization (e. g., Northern blot analysis) and/or amplification (e.g., RT-PCR). Preferably quantitative or semi-quantitative RT-PCR is preferred. Real-time quantitative or semi-quantitative RT-PCR is particularly advantageous.

Other methods of Amplification include ligase chain reaction (LCR), transcription-mediated amplification (TMA), strand displacement amplification (SDA) and nucleic acid sequence based amplification (NASBA), quantitative new generation sequencing of RNA (NGS).

Nucleic acids (polynucleotides) comprising at least 10 nucleotides and exhibiting sequence complementarity or homology to the mRNA of interest herein find utility as hybridization probes or amplification primers. It is understood that such nucleic acids need not be completely identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e. g. avidin/biotin).

Probes typically comprise single-stranded nucleic acids of between 10 to 1000 nucleotides in length, for instance of between 10 and 800, more preferably of between 15 and 700, typically of between 20 and 500 nucleotides. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

Nucleic acids which may be used as primers or probes in the above amplification and detection method may be assembled as a kit. Such a kit includes consensus primers and molecular probes. A preferred kit also includes the components necessary to determine if amplification has occurred. A kit may also include, for example, PCR buffers and enzymes; positive control sequences, reaction control primers; and instructions for amplifying and detecting the specific sequences.

In a particular embodiment, the methods of the invention comprise the steps of providing total RNAs extracted from cumulus cells and subjecting the RNAs to amplification and hybridization to specific probes, more particularly by means of a quantitative or semi-quantitative RT-PCR.

Probes made using the disclosed methods can be used for nucleic acid detection, such as in situ hybridization (ISH) procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pinkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. J. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publications Nos. 2006/0246524; 2006/0246523, and 2007/0117153.

It will be appreciated by those of skill in the art that by appropriately selecting labelled probe-specific binding agent pairs, multiplex detection schemes can be produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can be labelled with a first hapten, such as biotin, while a second probe that corresponds to a second target sequence can be labelled with a second hapten, such as DNP. Following exposure of the sample to the probes, the bound probes can be detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn). Additional probes/binding agent pairs can be added to the multiplex detection scheme using other spectrally distinct fluorophores. Numerous variations of direct, and indirect (one step, two step or more) can be envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In a particular embodiment, the methods of the invention comprise the steps of providing total RNAs extracted from cumulus cells and subjecting the RNAs to amplification and hybridization to specific probes, more particularly by means of a quantitative or semi-quantitative RT-PCR.

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

The expression level of a gene may be expressed as absolute expression level or normalized expression level. Both types of values may be used in the present method. The expression level of a gene is preferably expressed as normalized expression level when quantitative PCR is used as method of assessment of the expression level because small differences at the beginning of an experiment could provide huge differences after a number of cycles.

Typically, expression levels are normalized by correcting the absolute expression level of a gene by comparing its expression to the expression of a gene that is not relevant for determining the cancer stage of the patient, e.g., a housekeeping gene that is constitutively expressed. Suitable genes for normalization include housekeeping genes such as the actin gene ACTB, ribosomal 18S gene, GUSB, PGK1 and TFRC. This normalization allows comparing the expression level of one sample, e.g., a patient sample, with the expression level of another sample, or comparing samples from different sources.

Predetermined reference values used for comparison may consist of “cut-off” values that may be determined as described hereunder. Typically, the predetermined reference value (“cut-off”) may be determined by carrying out a method comprising the steps of:

a) providing a collection of tumor tissue samples from patients suffering of breast cancer;

b) determining the expression level of Vangl2 for each tumour tissue sample contained in the collection provided at step a);

c) ranking the tumor tissue samples according to said expression level

d) classifying said tumour tissue samples in pairs of subsets of increasing, respectively decreasing, number of members ranked according to their expression level,

e) providing, for each tumour tissue sample provided at step a), information relating to the actual clinical outcome for the corresponding cancer patient (i.e. the duration of the disease-free survival (DFS) or the overall survival (OS) or both);

f) for each pair of subsets of tumour tissue samples, obtaining a Kaplan Meier percentage of survival curve;

g) for each pair of subsets of tumour tissue samples calculating the statistical significance (p value) between both subsets

h) selecting as reference value for the expression level, the value of expression level for which the p value is the smallest.

A confidence interval may be constructed around the value of expression level thus obtained, for example ELR±5 or 10%.

For example the expression level of Vangl2 has been assessed for 100 cancer samples of 100 patients. The 100 samples are ranked according to the expression level of Vangl2. Sample 1 has the highest expression level and sample 100 has the lowest expression level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding cancer patient, Kaplan Meier curves are prepared for each of the 99 groups of two subsets. Also for each of the 99 groups, the p value between both subsets was calculated.

The predetermined reference value is selected such as the discrimination based on the criterion of the minimum p value is the strongest. In other terms, the expression level corresponding to the boundary between both subsets for which the p value is minimum is considered as the reference value. It should be noted that according to the experiments made by the inventors, the reference value is not necessarily the median value of expression levels.

In routine work, the reference value (cut-off value) may be used in the present method to discriminate tumour samples and therefore the corresponding patients.

Kaplan-Meier curves of percentage of survival as a function of time are commonly used to measure the fraction of patients living for a certain amount of time after treatment and are well known by the man skilled in the art. P value is conventionally used in statistical significance testing.

The man skilled in the art also understands that the same technique of assessment of the expression level of a gene should preferably be used for obtaining the reference value and thereafter for assessment of the expression level of a gene of a patient subjected to the method of the invention.

Of particular note is the fact that according to the technique of assessment of the expression level, a numerical value lower than the reference value may actually mean that the expression level is higher than the reference level. For example, in the examples thereafter, using real-time PCR, a dCt value lower than the relevant reference value means that the signal was detected earlier, i.e.: the expression level of the gene is higher than the reference level.

The method of the invention allows making a good assessment of prognosis with respect to DFS and OS of a patient.

The setting of a single “cut-off” value allows discrimination between a poor and a good prognosis with respect to DFS and OS for a patient. Practically, high statistical significance values (e.g. low P values) are generally obtained for a range of successive arbitrary quantification values, and not only for a single arbitrary quantification value. Thus, in one alternative embodiment of the invention, instead of using a definite reference value, a range of values is provided.

Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P value) are retained, so that a range of quantification values is provided. This range of quantification values includes a “cut-off” value as described above. According to this specific embodiment of a “cut-off” value, poor or good clinical outcome prognosis can be determined by comparing the expression level with the range of values which are identified. In certain embodiments, a cut-off value thus consists of a range of quantification values, e.g. centered on the quantification value for which the highest statistical significance value is found (e.g. generally the minimum P value which is found). For example, on a hypothetical scale of 1 to 10, if the ideal cut-off value (the value with the highest statistical significance) is 5, a suitable (exemplary) range may be from 4-6. Therefore, a patient may be assessed by comparing values obtained by measuring the expression level of Vangl2, where values greater than 5 reveal a poor prognosis and values less than 5 reveal a good prognosis. In a another embodiment, a patient may be assessed by comparing values obtained by measuring the expression level of Vangl2 and comparing the values on a scale, where values above the range of 4-6 indicate a poor prognosis and values below the range of 4-6 indicate a good prognosis, with values falling within the range of 4-6 indicating an intermediate prognosis.

According to another embodiment of the invention, the method for predicting the survival time further comprises the step of concluding that a patient would advantageously receive an antitumoral treatment when it is concluded that the patient has a poor prognosis.

In particular, the method for predicting the survival time further comprises the step of administering the patient with an anti-Vangl2 antibody when it is concluded that the patient has a poor prognosis.

In a particular embodiment, an anti-Vangl2 monoclonal antibody of the invention is used to induce antibody dependent cellular cytotoxicity (ADCC) or complement dependent cytotoxicity (CDC) against Vangl2-expressing cells. In another particular embodiment, the anti-Vangl2 antibody may be suitable for disturbing the expression of Vangl2 at the cell surface (e.g. by provoking internalization of Vangl2) so that cell migration, cell proliferation and tumour growth of tumor cells will be limited or inhibited.

The invention embraces antibodies or fragments of anti-Vangl2 antibodies.

In one embodiment, the antibodies or fragment of antibodies are directed to all or a portion of the extracellular domain of Vangl2. In one embodiment, the antibodies or fragment of antibodies are directed to an extracellular domain of Vangl2.

The term “antibody” is thus used to refer to any antibody-like molecule that has an antigen binding region, and this term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments.

In one embodiment of the antibodies or portions thereof described herein, the antibody is a monoclonal antibody. In one embodiment of the antibodies or portions thereof described herein, the antibody is a polyclonal antibody. In one embodiment of the antibodies or portions thereof described herein, the antibody is a humanized antibody. In one embodiment of the antibodies or portions thereof described herein, the antibody is a chimeric antibody. In one embodiment of the antibodies or portions thereof described herein, the portion of the antibody comprises a light chain of the antibody. In one embodiment of the antibodies or portions thereof described herein, the portion of the antibody comprises a heavy chain of the antibody. In one embodiment of the antibodies or portions thereof described herein, the portion of the antibody comprises a Fab portion of the antibody. In one embodiment of the antibodies or portions thereof described herein, the portion of the antibody comprises a F(ab′)2 portion of the antibody. In one embodiment of the antibodies or portions thereof described herein, the portion of the antibody comprises a Fc portion of the antibody. In one embodiment of the antibodies or portions thereof described herein, the portion of the antibody comprises a Fv portion of the antibody. In one embodiment of the antibodies or portions thereof described herein, the portion of the antibody comprises a variable domain of the antibody. In one embodiment of the antibodies or portions thereof described herein, the portion of the antibody comprises one or more CDR domains of the antibody.

Monoclonal antibodies may be generated using the method of Kohler and Milstein (Nature, 256:495, 1975). To prepare monoclonal antibodies useful in the invention, a mouse or other appropriate host animal is immunized at suitable intervals (e.g., twice-weekly, weekly, twice-monthly or monthly) with antigenic forms of Vangl2. The animal may be administered a final “boost” of antigen within one week of sacrifice. It is often desirable to use an immunologic adjuvant during immunization. Suitable immunologic adjuvants include Freund's complete adjuvant, Freund's incomplete adjuvant, alum, Ribi adjuvant, Hunter's Titermax, saponin adjuvants such as QS21 or Quil A, or CpG-containing immunostimulatory oligonucleotides. Other suitable adjuvants are well-known in the field. The animals may be immunized by subcutaneous, intraperitoneal, intramuscular, intravenous, intranasal or other routes. A given animal may be immunized with multiple forms of the antigen by multiple routes.

Briefly, the recombinant Vangl2 may be provided by expression with recombinant cell lines. Vangl2 may be provided in the form of human cells expressing Vangl2 at their surface. Recombinant forms of Vangl2 may be provided using any previously described method. Following the immunization regimen, lymphocytes are isolated from the spleen, lymph node or other organ of the animal and fused with a suitable myeloma cell line using an agent such as polyethylene glycol to form a hydridoma. Following fusion, cells are placed in media permissive for growth of hybridomas but not the fusion partners using standard methods, as described (Coding, Monoclonal Antibodies: Principles and Practice: Production and Application of Monoclonal Antibodies in Cell Biology, Biochemistry and Immunology, 3rd edition, Academic Press, New York, 1996). Following culture of the hybridomas, cell supernatants are analyzed for the presence of antibodies of the desired specificity, i.e., that selectively bind the antigen. Suitable analytical techniques include ELISA, flow cytometry, immunoprecipitation, and western blotting. Other screening techniques are well-known in the field. Preferred techniques are those that confirm binding of antibodies to conformationally intact, natively folded antigen, such as non-denaturing ELISA, flow cytometry, and immunoprecipitation.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The Fc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDRS). The CDRs, and in particular the CDRS regions, and more particularly the heavy chain CDRS, are largely responsible for antibody specificity.

It is now well-established in the art that the non CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

This invention provides in certain embodiments compositions and methods that include humanized forms of antibodies. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference. The above U.S. Pat. Nos. 5,585,089 and 5,693,761, and WO 90/07861 also propose four possible criteria which may used in designing the humanized antibodies. The first proposal was that for an acceptor, use a framework from a particular human immunoglobulin that is unusually homologous to the donor immunoglobulin to be humanized, or use a consensus framework from many human antibodies. The second proposal was that if an amino acid in the framework of the human immunoglobulin is unusual and the donor amino acid at that position is typical for human sequences, then the donor amino acid rather than the acceptor may be selected. The third proposal was that in the positions immediately adjacent to the 3 CDRs in the humanized immunoglobulin chain, the donor amino acid rather than the acceptor amino acid may be selected. The fourth proposal was to use the donor amino acid reside at the framework positions at which the amino acid is predicted to have a side chain atom within 3A of the CDRs in a three dimensional model of the antibody and is predicted to be capable of interacting with the CDRs. The above methods are merely illustrative of some of the methods that one skilled in the art could employ to make humanized antibodies. One of ordinary skill in the art will be familiar with other methods for antibody humanization.

In one embodiment of the humanized forms of the antibodies, some, most or all of the amino acids outside the CDR regions have been replaced with amino acids from human immunoglobulin molecules but where some, most or all amino acids within one or more CDR regions are unchanged. Small additions, deletions, insertions, substitutions or modifications of amino acids are permissible as long as they would not abrogate the ability of the antibody to bind a given antigen. Suitable human immunoglobulin molecules would include IgG1, IgG2, IgG3, IgG4, IgA and IgM molecules. A “humanized” antibody retains a similar antigenic specificity as the original antibody. However, using certain methods of humanization, the affinity and/or specificity of binding of the antibody may be increased using methods of “directed evolution”, as described by Wu et al., /. Mol. Biol. 294:151, 1999, the contents of which are incorporated herein by reference.

Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference. These animals have been genetically modified such that there is a functional deletion in the production of endogenous (e.g., murine) antibodies. The animals are further modified to contain all or a portion of the human germ-line immunoglobulin gene locus such that immunization of these animals will result in the production of fully human antibodies to the antigen of interest. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (KAMA) responses when administered to humans.

In vitro methods also exist for producing human antibodies. These include phage display technology (U.S. Pat. Nos. 5,565,332 and 5,573,905) and in vitro stimulation of human B cells (U.S. Pat. Nos. 5,229,275 and 5,567,610). The contents of these patents are incorporated herein by reference.

Thus, as will be apparent to one of ordinary skill in the art, the present invention also provides for F(ab′) 2 Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The various antibody molecules and fragments may derive from any of the commonly known immunoglobulin classes, including but not limited to IgA, secretory IgA, IgE, IgG and IgM. IgG subclasses are also well known to those in the art and include but are not limited to human IgG1, IgG2, IgG3 and IgG4.

It may be also desirable to modify the antibody of the invention with respect to effector function, e.g. so as to enhance antigen-dependent cell-mediated cytotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing inter-chain disulfide bond formation in this region. The homodimeric antibody thus generated may have improved internalization capability and/or increased complement-mediated cell killing and/or antibody-dependent cellular cytotoxicity (ADCC) (Caron PC. et al. 1992; and Shopes B. 1992)

In another aspect, the present invention provides an anti-Vangl2 monoclonal antibody-drug conjugate. An “anti-Vangl2 monoclonal antibody-drug conjugate” as used herein refers to an anti-Vangl2 monoclonal antibody according to the invention conjugated to a therapeutic agent. Such anti-Vangl2 monoclonal antibody-drug conjugates produce clinically beneficial effects on Vangl2-expressing tumor cells when administered to a subject.

In typical embodiments, an anti-Vangl2 monoclonal antibody is conjugated to a cytotoxic agent, such that the resulting antibody-drug conjugate exerts a cytotoxic or cytostatic effect on a Vangl2-expressing tumor cell when taken up or internalized by the cell. Particularly suitable moieties for conjugation to antibodies are chemotherapeutic agents, prodrug converting enzymes, radioactive isotopes or compounds, or toxins. For example, an anti-Vangl2 monoclonal antibody can be conjugated to a cytotoxic agent such as a chemotherapeutic agent or a toxin (e.g., a cytostatic or cytocidal agent such as, for example, saporin, abrin, ricin A, pseudomonas exotoxin, or diphtheria toxin).

Useful classes of cytotoxic agents include, for example, antitubulin agents, auristatins, DNA minor groove binders, DNA replication inhibitors, alkylating agents (e.g., platinum complexes such as cis-platin, mono(platinum), bis(platinum) and tri-nuclear platinum complexes and-carboplatin), anthracyclines, antibiotics, antifolates, antimetabolites, chemotherapy sensitizers, duocarmycins, etoposides, fluorinated pyrimidines, ionophores, lexitropsins, nitrosoureas, platinols, pre-forming compounds, purine antimetabolites, puromycins, radiation sensitizers, steroids, taxanes, topoisomerase inhibitors, vinca alkaloids, or the like.

Individual cytotoxic agents include, for example, an androgen, anthramycin (AMC), asparaginase, 5-azacytidine, azathioprine, bleomycin, busulfan, buthionine sulfoximine, camptothecin, carboplatin, carmustine (BSNU), CC-1065 (Li et al., Cancer Res. 42:999-1004, 1982), chlorambucil, cisplatin, colchicine, cyclophosphamide, cytarabine, cytidine arabinoside, cytochalasin B, dacarbazine, dactinomycin (formerly actinomycin), daunorubicin, decarbazine, docetaxel, doxorubicin, an estrogen, 5-fluordeoxyuridine, etopside phosphate (VP-16), 5-fluorouracil, gramicidin D, hydroxyurea, idarubicin, ifosfamide, irinotecan, lomustine (CCNU), mechlorethamine, melphalan, 6-mercaptopurine, methotrexate, mithramycin, mitomycin C, mitoxantrone, nitroimidazole, paclitaxel, plicamycin, procarbizine, streptozotocin, tenoposide (VM-26), 6-thioguanine, thioTEPA, topotecan, vinblastine, vincristine, and vinorelbine.

Particularly suitable cytotoxic agents include, for example, dolastatins (e.g., auristatin E, AFP, MMAF, MMAE), DNA minor groove binders (e.g., enediynes and lexitropsins), duocarmycins, taxanes (e.g., paclitaxel and docetaxel), puromycins, vinca alkaloids, CC-1065, SN-38 (7-ethyl-10-hydroxy-camptothein), topotecan, morpholino-doxorubicin, rhizoxin, cyanomorpholino-doxorubicin, echinomycin, combretastatin, netropsin, epothilone A and B, estramustine, cryptophysins, cemadotin, maytansinoids, discodermolide, eleutherobin, and mitoxantrone.

In certain embodiments, a cytotoxic agent is a conventional chemotherapeutic such as, for example, doxorubicin, paclitaxel, melphalan, vinca alkaloids, methotrexate, mitomycin C or etoposide. In addition, potent agents such as CC-1065 analogues, calicheamicin, maytansine, analogues of dolastatin 10, rhizoxin, and palytoxin can be linked to an anti-Vang12-expressing antibody.

In specific variations, the cytotoxic or cytostatic agent is auristatin E (also known in the art as dolastatin-10) or a derivative thereof. Typically, the auristatin E derivative is, e.g., an ester formed between auristatin E and a keto acid. For example, auristatin E can be reacted with paraacetyl benzoic acid or benzoylvaleric acid to produce AEB and AEVB, respectively. Other typical auristatin derivatives include AFP (dimethylvaline-valine-dolaisoleuine-dolaproine-phenylalanine-p-phenylenediamine), MMAF (dovaline-valine-dolaisoleunine-dolaproine-phenylalanine), and MAE (monomethyl auristatin E). The synthesis and structure of auristatin E and its derivatives are described in U.S. Patent Application Publication No. 20030083263; International Patent Publication Nos. WO 2002/088172 and WO 2004/010957; and U.S. Pat. Nos. 6,884,869; 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414.

In other variations, the cytotoxic agent is a DNA minor groove binding agent. (See, e.g., U.S. Pat. No. 6,130,237.) For example, in certain embodiments, the minor groove binding agent is a CBI compound. In other embodiments, the minor groove binding agent is an enediyne (e.g., calicheamicin).

In certain embodiments, an antibody-drug conjugate comprises an anti-tubulin agent. Examples of anti-tubulin agents include, for example, taxanes (e.g., Taxol® (paclitaxel), Taxotere® (docetaxel)), T67 (Tularik), vinca alkyloids (e.g., vincristine, vinblastine, vindesine, and vinorelbine), and dolastatins (e.g., auristatin E, AFP, MMAF, MMAE, AEB, AEVB). Other antitubulin agents include, for example, baccatin derivatives, taxane analogs (e.g., epothilone A and B), nocodazole, colchicine and colcimid, estramustine, cryptophysins, cemadotin, maytansinoids, combretastatins, discodermolide, and eleutherobin. In some embodiments, the cytotoxic agent is a maytansinoid, another group of anti-tubulin agents. For example, in specific embodiments, the maytansinoid is maytansine or DM-1 (ImmunoGen, Inc.; see also Chari et al., Cancer Res. 52:127-131, 1992).

In other embodiments, the cytotoxic agent is an antimetabolite. The antimetabolite can be, for example, a purine antagonist (e.g., azothioprine or mycophenolate mofetil), a dihydrofolate reductase inhibitor (e.g., methotrexate), acyclovir, gangcyclovir, zidovudine, vidarabine, ribavarin, azidothymidine, cytidine arabinoside, amantadine, dideoxyuridine, iododeoxyuridine, poscarnet, or trifluridine.

In other embodiments, an anti-Vangl2 monoclonal antibody is conjugated to a pro-drug converting enzyme. The pro-drug converting enzyme can be recombinantly fused to the antibody or chemically conjugated thereto using known methods. Exemplary pro-drug converting enzymes are carboxypeptidase G2, beta-glucuronidase, penicillin-V-amidase, penicillin-G-amidase, beta-lactamase, beta-glucosidase, nitroreductase and carboxypeptidase A.

Techniques for conjugating therapeutic agents to proteins, and in particular to antibodies, are well-known. (See, e.g., Amon et al., “Monoclonal Antibodies For Immunotargeting Of Drugs In Cancer Therapy,” in Monoclonal Antibodies And Cancer Therapy (Reisfeld et al. eds., Alan R. Liss, Inc., 1985); Hellstrom et al., “Antibodies For Drug Delivery,” in Controlled Drug Delivery (Robinson et al. eds., Marcel Deiker, Inc., 2nd ed. 1987); Thorpe, “Antibody Carriers Of Cytotoxic Agents In Cancer Therapy: A Review,” in Monoclonal Antibodies '84: Biological And Clinical Applications (Pinchera et al. eds., 1985); “Analysis, Results, and Future Prospective of the Therapeutic Use of Radiolabeled Antibody In Cancer Therapy,” in Monoclonal Antibodies For Cancer Detection And Therapy (Baldwin et al. eds., Academic Press, 1985); and Thorpe et al., 1982, Immunol. Rev. 62:119-58. See also, e.g., PCT publication WO 89/12624.)

In a particular embodiment, an anti-Vangl2 monoclonal antibody of the invention is used to induce antibody dependent cellular cytotoxicity (ADCC). In ADCC, monoclonal antibodies bind to a target cell (e.g., cancer cell) and specific effector cells expressing receptors for the monoclonal antibody (e.g., NK cells, CD8+ T cells, monocytes, granulocytes) bind the monoclonal antibody/target cell complex resulting in target cell death.

Accordingly, in some embodiments, an anti-Vangl2 monoclonal antibody comprising an Fc region with effector function is used to induce antibody dependent cellular cytotoxicity (ADCC) or complement dependent cytotoxicity (CDC) against a Vangl2-expressing cell. Methods for inducing ADCC generally include contacting the Vangl2-expressing cell with an effective amount an anti-Vangl2 monoclonal antibody comprising an Fc region having ADCC activity, wherein the contacting step is in the presence of a cytolytic immune effector cell expressing an Fc receptor having cytolytic activity. Immune effector cells expressing cytolytic Fc receptors (e.g., FcγRIIIα or CD16) include, for example, NK cells as well certain CD8+ T cells. Methods for inducing CDC generally include contacting the Vangl2-expressing cell with an effective amount an anti-Vangl2 monoclonal antibody comprising an Fc region having CDC activity, wherein the contacting step is in the presence of complement.

In related embodiments, an anti-Vangl2 monoclonal antibody comprising an Fc region with effector function, as described herein, is used to treat the patient. Such methods generally include administering to a subject an effective amount of an anti-Vangl2 monoclonal antibody comprising an Fc region having ADCC activity.

In another embodiment, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “nanobody®”. According to the invention, sdAb can particularly be llama sdAb.

In some embodiments, the antibodies can be monospecific, bispecific, trispecific, or of greater multispecificity. Multispecific antibodies, including bispecific and trispecific antibodies, useful for practicing the methods described herein are antibodies that immunospecifically bind to both Vangl2 and a second cell surface receptor or receptor complex that mediates ADCC, phagocytosis, and/or CDC, such as CD16/FcgRIII, CD64/FcgRI, killer inhibitory or activating receptors, or the complement control protein CD59. In a typical embodiment, the binding of the portion of the multispecific antibody to the second cell surface molecule or receptor complex enhances the effector functions of the anti-Vang12 antibody or other Vangl2 binding agent. In some embodiment, the anti-Vangl2 antibody is a bispecific antibody. The term “bispecific antibody” has its general meaning in the art and refers to any molecule consisting of one binding site for a target antigen on tumor cells and a second binding side for an activating trigger molecule on an effector cell, such as CD3 on T-cells, CD16 (FcyRIII) on natural killer (NK) cells, monocytes and macrophages, CD89 (FcαRI) and CD64 (FcyRI) on neutrophils and monocytes/macrophages, and DEC-205 on dendritic cells. According to the invention, the bispecific antibody comprises a binding site for Vangl2. tApart from the specific recruitment of the preferred effector cell population, bispecific antibodies avoid competition with endogenous immunoglobulin G (IgG) when the selected binding site for the trigger molecule on the effector cell does not overlap with Fc-binding epitopes. In addition, the use of single-chain Fv fragments instead of full-length immunoglobulin prevents the molecules from binding to Fc-receptors on non-cytotoxic cells, such as FcγRII on platelets and B-cells, to Fc-receptors that do not activate cytotoxic cells, including FcyRIIIb on polymorphonuclear leukocytes (PMN), and to inhibitory Fc-receptors, such as FcyRIIb on monocytes/macrophages. Methods for making bispecific antibodies are known in the art. Traditional production of full-length bispecific antibodies is based on the coexpression of two immunoglobulin heavy chain-light chain pairs, where the two chains have different specificities (see, e.g., Milstein et al., 1983, Nature 305:537-39). Because of the random assortment of immunoglobulin heavy and light chains, these hybridomas (quadromas) produce a potential mixture of 10 different antibody molecules, of which only one has the correct bispecific structure. Similar procedures are disclosed in International Publication No. WO 93/08829, and in Traunecker et al., 1991, EMBO J. 10:3655-59. Other examples of bispecific antibodies include Bi-specific T-cell engagers (BiTEs) that are a class of artificial bispecific monoclonal antibodies. BiTEs are fusion proteins consisting of two single-chain variable fragments (scFvs) of different antibodies, or amino acid sequences from four different genes, on a single peptide chain of about 55 kilodaltons. One of the scFvs binds to tumor antigen (i.e. Vangl2) and the other generally to the a n effector cell (e.g. a T cell via the CD3 receptor. Other bispecific antibodies those described in WO2006064136. In particular the bispecific antibody is a Fab format described in WO2006064136 comprising one VH or VHH specific for Vangl2 and one VH or VHH specific for an effector cell.

In particular, the method for predicting the survival time further comprises the step of administering the patient with an inhibitor of Vangl2 expression when it is concluded that the patient has a poor prognosis.

An “inhibitor of expression” refers to a natural or synthetic compound that has a biological effect to inhibit the expression of a gene. Therefore, an “inhibitor of Vangl2 expression” denotes a natural or synthetic compound that has a biological effect to inhibit the expression of Vangl2 gene.

In a preferred embodiment of the invention, said inhibitor of gene expression is a siRNA, an antisense oligonucleotide or a ribozyme.

Inhibitors of gene expression for use in the present invention may be based on antisense oligonucleotide constructs. Anti-sense oligonucleotides, including anti-sense RNA molecules and anti-sense DNA molecules, would act to directly block the translation of Vangl2 mRNA by binding thereto and thus preventing protein translation or increasing mRNA degradation, thus decreasing the level of Vangl2, and thus activity, in a cell. For example, antisense oligonucleotides of at least about 15 bases and complementary to unique regions of the mRNA transcript sequence encoding Vangl2 can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of gene expression for use in the present invention. Gene expression can be reduced by contacting the tumor, subject or cell with a small double stranded RNA (dsRNA), or a vector or construct causing the production of a small double stranded RNA, such that gene expression is specifically inhibited (i.e. RNA interference or RNAi). Methods for selecting an appropriate dsRNA or dsRNA-encoding vector are well known in the art for genes whose sequence is known (e.g. see Tuschi, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of Vangl2 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Cliffton, N.J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hematopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In particular, the method for predicting the survival time further comprises the step of administering the patient with an aptamer directed against Vangl2 when it is concluded that the patient has a poor prognosis. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library. The random sequence library is obtainable by combinatorial chemical synthesis of DNA. In this library, each member is a linear oligomer, eventually chemically modified, of a unique sequence.

In particular, the method for predicting the survival time further comprises the step of administering the patient with an activator of autophagy when it is concluded that the patient has a poor prognosis.

The term “activator of autophagy” denotes any compound natural or not that is able to induce autophagy in a cell. Typically, the activator of autophagy is a mTOR inhibitor.

The term “mTOR inhibitor” as used herein refers to any compound capable of inhibiting the expression and/or activity of the mammalian target of rapamycin (mTOR) protein (also known as FK506 binding protein 12-rapamycin associated protein 1 (FRAP1)), and more particularly of the mTOR Complex 1 (mTORCI). MTORC1 comprises at least four proteins, namely mTOR, regulatory associated protein of mTOR (Raptor), mammalian LST8/G-protein β-subunit like protein (mLST8/Gβ1_) and proline-rich Akt substrate of 40 kDa (PRAS40).

A representative mTOR inhibitor is the macrolide rapamycin (also known as sirolimus, Rapamune™, which is a product of Streptomyces hygroscopicus.

mTOR inhibitors also include any analog, derivative, prodrug or metabolite of rapamycin, such as esters, ethers, oximes, hydrazones, and hydroxylamines of rapamycin, as well as rapamycins in which functional groups on the rapamycin nucleus have been modified, for example through reduction or oxidation. Esters and ethers of rapamycin include, for example, alkyl esters (U.S. Pat. No. 4,316,885); aminoalkyl esters (U.S. Pat. No. 4,650,803); fluorinated esters (U.S. Pat. No. 5,100,883); amide esters (U.S. Pat. No. 5,118,677); carbamate esters (U.S. Pat. No. 5,118,678); silyl ethers (U.S. Pat. No. 5,120,842); aminoesters (U.S. Pat. No. 5,130,307); aminodiesters (U.S. Pat. No. 5,162,333); sulfonate and sulfate esters (U.S. Pat. No. 5,177,203); esters (U.S. Pat. No. 5,221,670); alkoxyesters (U.S. Pat. No. 5,233,036); O-aryl, -alkyl, -alkenyl, and -alkynyl ethers (U.S. Pat. No. 5,258,389); carbonate esters (U.S. Pat. No. 5,260,300); arylcarbonyl and alkoxycarbonyl carbamates (U.S. Pat. No. 5,262,423); carbamates (U.S. Pat. No. 5,302,584); hydroxyesters (U.S. Pat. No. 5,362,718); hindered esters (U.S. Pat. No. 5,385,908); heterocyclic esters (U.S. Pat. No. 5,385,909); gem-disubstituted esters (U.S. Pat. No. 5,385,910); amino alkanoic esters (U.S. Pat. No. 5,389,639); phosphorylcarbamate esters (U.S. Pat. No. 5,391,730); carbamate esters (U.S. Pat. Nos. 5,411,967, 5,434,260, 5,480,988, 5,480,989 and 5,489,680); hindered N-oxide esters (U.S. Patent 5,491,231); biotin esters (U.S. Pat. No. 5,504,091); O-alkyl ethers (U.S. Pat. No. 5,665,772); and PEG esters of rapamycin (U.S. Pat. No. 5,780,462). The preparation of these esters and ethers are disclosed in the patents listed above. Oximes, hydrazones, and hydroxylamines of rapamycin are disclosed, for example, in U.S. Pat. Nos. 5,373,014, 5,378,836, 5,023,264, and 5,563,145. The preparation of these oximes, hydrazones, and hydroxylamines are disclosed in the above listed patents.

In an embodiment, the above-mentioned rapamycin derivative is Everolimus (also known as RAD-001, Certican™, and Afinitor™) or Temsirolimus (also known as CCI-779 and Torisel™). In a further embodiment, a combination of mTOR inhibitors may be used, such as a combination of rapamycin derivatives, for example a combination of Everolimus and Temsirolimus.

The anti-Vangl2 antibody, the anti-Vangl2 aptamer, the inhibitor of Vangl2 expression, or the activator of autophagy (e.g. the mTOR inhibitor) is administered to the patient in a therapeutically effective amount.

By a “therapeutically effective amount” of the antibody of the invention is meant a sufficient amount of the antibody to treat said cancer, at a reasonable benefit/risk ratio applicable to any medical treatment. It will be understood, however, that the total daily usage of the antibodies and compositions of the present invention will be decided by the attending physician within the scope of sound medical judgment. The specific therapeutically effective dose level for any particular patient will depend upon a variety of factors including the disorder being treated and the severity of the disorder; activity of the specific antibody employed; the specific composition employed, the age, body weight, general health, sex and diet of the patient; the time of administration, route of administration, and rate of excretion of the specific antibody employed; the duration of the treatment; drugs used in combination or coincidental with the specific antibody employed; and like factors well known in the medical arts. For example, it is well known within the skill of the art to start doses of the compound at levels lower than those required to achieve the desired therapeutic effect and to gradually increase the dosage until the desired effect is achieved.

In certain embodiments, the anti-Vangl2 monoclonal antibody, the anti-Vangl2 aptamer, the inhibitor of Vangl2 expression, or the activator of autophagy (e.g. the mTOR inhibitor) is used in combination with conventional cancer therapies such as, e.g., surgery, radiotherapy, chemotherapy, or combinations thereof. In certain aspects, other therapeutic agents useful for combination cancer therapy with an anti-Vangl2 antibody.

For administration, the anti-Vangl2 monoclonal antibody, the anti-Vangl2 aptamer, the inhibitor of Vangl2 expression, or the activator of autophagy (e.g. the mTOR inhibitor) is formulated as a pharmaceutical composition. A pharmaceutical composition comprising an anti-Vangl2 monoclonal antibody can be formulated according to known methods to prepare pharmaceutically useful compositions, whereby the therapeutic molecule is combined in a mixture with a pharmaceutically acceptable carrier. A composition is said to be a “pharmaceutically acceptable carrier” if its administration can be tolerated by a recipient patient. Sterile phosphate-buffered saline is one example of a pharmaceutically acceptable carrier. Other suitable carriers are well-known to those in the art. (See, e.g., Gennaro (ed.), Remington's Pharmaceutical Sciences (Mack Publishing Company, 19th ed. 1995).) Formulations may further include one or more excipients, preservatives, solubilizers, buffering agents, albumin to prevent protein loss on vial surfaces, etc.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, parenteral, intranasal, intravenous, intramuscular, subcutaneous or intraocular administration and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

To prepare pharmaceutical compositions, an effective amount of the antibody may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

An antibody of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The antibodies of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds formulated for parenteral administration, such as intravenous or intramuscular injection, other pharmaceutically acceptable forms include, e.g. tablets or other solids for oral administration; time release capsules; and any other form currently used.

In certain embodiments, the use of liposomes and/or nanoparticles is contemplated for the introduction of antibodies into host cells. The formation and use of liposomes and/or nanoparticles are known to those of skill in the art.

Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles that meet these requirements are contemplated for use in the present invention, and such particles may be are easily made.

Liposomes are formed from phospho lipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core. The physical characteristics of liposomes depend on pH, ionic strength and the presence of divalent cations.

A further aspect of the invention consists of a method for screening a drug for the treatment of breast cancer comprising the steps consisting of a) determining the ability of a candidate compound to inhibit the interaction between a Vangl2 polypeptide and a p62 polypeptide and b) positively selecting the candidate compound that inhibits said interaction.

The method is particularly suitable for screening a drug for the treatment of basal breast cancer, a metastatic breast cancer or a triple negative breast cancer.

At step a), any method suitable for the screening of protein-protein interactions is suitable.

Whatever the embodiment of step a) of the screening method, the complete Vangl2 protein and the complete p62 protein may be used as the binding partners. Alternatively, fragments of Vangl2 protein and p62 protein that include the site of interaction may be used as the binding partners.

Therefore in one embodiment step a) of the screening method of the invention consists of the following steps:
a1) bringing into contact the candidate compound to be tested with a mixture of a first Vangl2 polypeptide or a substantially homologous or substantially similar amino acid sequence thereof and (2) a second p62 polypeptide or a substantially homologous or substantially similar amino acid sequence thereof
a2) determining the ability of said candidate compound to modulate the binding between said Vangl2 polypeptide and said second p62 polypeptide.

The term “polypeptide” means herein a polymer of amino acids having no specific length. Thus, peptides, oligopeptides and proteins are included in the definition of “polypeptide” and these terms are used interchangeably throughout the specification, as well as in the claims. The term “polypeptide” does not exclude post-translational modifications that include but are not limited to phosphorylation, acetylation, glycosylation and the like. Especially, the term includes all phosphorylated forms of the polypeptide (e.g. all phosphorylated forms of Vangl2 or p62). Also encompassed by this definition of “polypeptide” are homologs thereof.

Accordingly, the term “Vangl2 polypeptide” refers to the Vangl2 protein or a fragment thereof that comprises the site of interaction with p62 protein. Thus a Vangl2 polypeptide comprises the C-terminal Vangl2 region, i.e., the domain ranging from the residue at position 242 to the residue at position 521 of SEQ ID NO:1.

As used herein, the term “p62” refers to p62/sequestosome-1. An exemplary amino acid sequence is set forth as SEQ ID NO:2:

(p62_homo sapiens)
SEQ ID NO: 2
MASLTVKAYLLGKEDAAREIRRFSFCCSPEPEAEAEAAAGPGPCERLLS
RVAALFPALRPGGFQAHYRDEDGDLVAFSSDEELTMAMSYVKDDIFRIY
IKEKKECRRDHRPPCAQEAPRNMVHPNVICDGCNGPVVGTRYKCSVCPD
YDLCSVCEGKGLHRGHTKLAFPSPFGHLSEGFSHSRWLRKVKHGHFGWP
GWEMGPPGNWSPRPPRAGEARPGPTAESASGPSEDPSVNFLKNVGESVA
AALSPLGIEVDIDVEHGGKRSRLTPVSPESSSTEEKSSSQPSSCCSDPS
KPGGNVEGATQSLAEQMRKTALESEGRPEEQMESDNCSGGDDDWTHLSS
KEVDPSTGELQSLQMPESEGPSSLDPSQEGPTGLKEAALYPHLPPEADP
RLIESLSQMLSMGFSDEGGWLTRLLQTKNYDIGAALDTIQYSKHPPPL

In the same manner, the term “p62 polypeptide” refers to the p62 protein or a fragment thereof that comprises the site of interaction with Vangl2 protein. Thus a p62 polypeptide comprises the domain ranging from the residue at position 346 to the residue at position 371 of SEQ ID NO:2. In particular, a p62 polypeptide comprises the domain ranging from the residue at position 346 to the residue at position 388 of SEQ ID NO:2.

Two amino acid sequences are “substantially homologous” or “substantially similar” when greater than 80%, preferably greater than 85%, preferably greater than 90% of the amino acids are identical, or greater than about 90%, preferably greater than 95%, are similar (functionally identical). The term “sequence identity” refers to the identity between two peptides. Identity between sequences can be determined by comparing a position in each of the sequences which may be aligned for the purposes of comparison. When a position in the compared sequences is occupied by the same base or amino acid, then the sequences are identical at that position. A degree of sequence identity between nucleic acid sequences is a function of the number of identical nucleotides at positions shared by these sequences. A degree of identity between amino acid sequences is a function of the number of identical amino acid sequences that are shared between these sequences. To determine the percent identity of two amino acids sequences or two nucleic acid sequences, the sequences are aligned for optimal comparison. For example, gaps can be introduced in the sequence of a first amino add sequence or a first nucleic acid sequence for optimal alignment with the second amino acid sequence or second nucleic acid sequence. The amino acid residues or nucleotides at corresponding amino acid positions or nucleotide positions are then compared. When a position in the first sequence is occupied by the same amino acid residue or nucleotide as the corresponding position in the second sequence, the molecules are identical at that position. The percent identity between the two sequences is a function of the number of identical positions shared by the sequences. In this comparison the sequences can be the same length or may be different in length. Optimal alignment of sequences for determining a comparison window may be conducted by the local homology algorithm of Smith and Waterman (J. Theor. Biol., 91 (2) pgs. 370-380 (1981), by the homology alignment algorithm of Needleman and Wunsch, J. Miol. Biol., 48(3) pgs. 443-453 (1972), by the search for similarity via the method of Pearson and Lipman, PNAS, USA, 85(5) pgs. 2444-2448 (1988), by computerized implementations of these algorithms (GAP, BESTFIT, FASTA and TFASTA in the Wisconsin Genetics Software Package Release 7.0, Genetic Computer Group, 575, Science Drive, Madison, Wis.) or by inspection. The term “sequence similarity” means that amino acids can be modified while retaining the same function. It is known that amino acids are classified according to the nature of their side groups and some amino adds such as the basic amino acids can be interchanged for one another while their basic function is maintained.

In one embodiment the step a2) consists in generating physical values which illustrate or not the ability of said candidate compound to inhibit the interaction between said first polypeptide and said second polypeptide and comparing said values with standard physical values obtained in the same assay performed in the absence of the said candidate compound. The “physical values” that are referred to above may be of various kinds depending of the binding assay that is performed, but notably encompass light absorbance values, radioactive signals and intensity value of fluorescence signal. If after the comparison of the physical values with the standard physical values, it is determined that the said candidate compound modulates the binding between said first polypeptide and said second polypeptide, then the candidate is positively selected at step b).

The compounds that inhibit the interaction between (i) the Vangl2 polypeptide and (ii) the p62 polypeptide encompass those compounds that bind either to the Vangl2 polypeptide or to p62 polypeptide, provided that the binding of the said compounds of interest then modulates the interaction between Vangl2 and p62.

Polypeptides of the invention may be produced by any technique known per se in the art, such as without limitation, any chemical, biological, genetic or enzymatic technique, either alone or in combination(s).

Knowing the amino acid sequence of the desired sequence, one skilled in the art can readily produce said polypeptides, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions.

Alternatively, the polypeptides of the invention can be synthesized by recombinant DNA techniques as is now well-known in the art. For example, these fragments can be obtained as DNA expression products after incorporation of DNA sequences encoding the desired (poly)peptide into expression vectors and introduction of such vectors into suitable eukaryotic or prokaryotic hosts that will express the desired polypeptide, from which they can be later isolated using well-known techniques.

A wide variety of host/expression vector combinations are employed in expressing the nucleic acids encoding for the polypeptides of the present invention. Useful expression vectors that can be used include, for example, segments of chromosomal, non-chromosomal and synthetic DNA sequences. Suitable vectors include, but are not limited to, derivatives of SV40 and pcDNA and known bacterial plasmids such as col EI, pCR1, pBR322, pMal-C2, pET, pGEX, pMB9 and derivatives thereof, plasmids such as RP4, phage DNAs such as the numerous derivatives of phage I such as NM989, as well as other phage DNA such as M13 and filamentous single stranded phage DNA; yeast plasmids such as the 2 microns plasmid or derivatives of the 2 microns plasmid, as well as centomeric and integrative yeast shuttle vectors; vectors useful in eukaryotic cells such as vectors useful in insect or mammalian cells; vectors derived from combinations of plasmids and phage DNAs, such as plasmids that have been modified to employ phage DNA or the expression control sequences; and the like.

Consequently, mammalian and typically human cells, as well as bacterial, yeast, fungi, insect, nematode and plant cells an used in the present invention and may be transfected by the nucleic acid or recombinant vector as defined herein. Examples of suitable cells include, but are not limited to, VERO cells, HELA cells such as ATCC No. CCL2, CHO cell lines such as ATCC No. CCL61, COS cells such as COS-7 cells and ATCC No. CRL 1650 cells, W138, BHK, HepG2, 3T3 such as ATCC No. CRL6361, A549, PC12, K562 cells, 293T cells, Sf9 cells such as ATCC No. CRL1711 and Cv1 cells such as ATCC No. CCL70. Other suitable cells that can be used in the present invention include, but are not limited to, prokaryotic host cells strains such as Escherichia coli, (e.g., strain DH5-[alpha]), Bacillus subtilis, Salmonella typhimurium, or strains of the genera of Pseudomonas, Streptomyces and Staphylococcus. Further suitable cells that can be used in the present invention include yeast cells such as those of Saccharomyces such as Saccharomyces cerevisiae.

In one embodiment, any Vangl2 derived or p62 polypeptide of the invention is labelled with a detectable molecule for the screening purposes.

According to the invention, said detectable molecule may consist of any compound or substance that is detectable by spectroscopic, photochemical, biochemical, immunochemical or chemical means. For example, useful detectable molecules include radioactive substance (including those comprising 32P, 25S, 3H, or 125I), fluorescent dyes (including 5-bromodesosyrudin, fluorescein, acetylaminofluorene or digoxigenin), fluorescent proteins (including GFPs and YFPs), or detectable proteins or peptides (including biotin, polyhistidine tails or other antigen tags like the HA antigen, the FLAG antigen, the c-myc antigen and the DNP antigen).

According to the invention, the detectable molecule is located at, or bound to, an amino acid residue located outside the said amino acid sequence of interest, in order to minimise or prevent any artefact for the binding between said polypeptides or between the candidate compound and or any of said polypeptides.

In another particular embodiment, the polypeptides of the invention are fused with a GST tag (Glutathione S-transferase). In this embodiment, the GST moiety of the said fusion protein may be used as detectable molecule. In the said fusion protein, the GST may be located either at the N-terminal end or at the C-terminal end. The GST detectable molecule may be detected when it is subsequently brought into contact with an anti-GST antibody, including with a labelled anti-GST antibody. Anti-GST antibodies labelled with various detectable molecules are easily commercially available.

In another particular embodiment, the polypeptides of the invention are fused with a poly-histidine tag. Said poly-histidine tag usually comprises at least four consecutive hisitidine residues and generally at least six consecutive histidine residues. Such a polypeptide tag may also comprise up to 20 consecutive histidine residues. Said poly-histidine tag may be located either at the N-terminal end or at the C-terminal end In this embodiment, the poly-histidine tag may be detected when it is subsequently brought into contact with an anti-poly-histidine antibody, including with a labelled anti-poly-histidine antibody. Anti-poly-histidine antibodies labelled with various detectable molecules are easily commercially available.

In a further embodiment, the polypeptides of the invention are fused with a protein moiety consisting of either the DNA binding domain or the activator domain of a transcription factor. Said protein moiety domain of transcription may be located either at the N-terminal end or at the C-terminal end. Such a DNA binding domain may consist of the well-known DNA binding domain of LexA protein originating form E. Coli. Moreover said activator domain of a transcription factor may consist of the activator domain of the well-known Ga14 protein originating from yeast.

In one embodiment of the screening method according to the invention, the first Vangl2 polypeptide and second p62 polypeptide as described above, comprise a portion of a transcription factor. In said assay, the binding together of the first and second portions generates a functional transcription factor that binds to a specific regulatory DNA sequence, which in turn induces expression of a reporter DNA sequence, said expression being further detected and/or measured. A positive detection of the expression of said reporter DNA sequence means that an active transcription factor is formed, due to the binding together of said first Vangl2 polypeptide and second p62 polypeptide polypeptide.

Usually, in a two-hybrid assay, the first and second portion of a transcription factor consist respectively of (i) the DNA binding domain of a transcription factor and (ii) the activator domain of a transcription factor. In some embodiments, the DNA binding domain and the activator domain both originate from the same naturally occurring transcription factor. In some embodiments, the DNA binding domain and the activator domain originate from distinct naturally occurring factors, while, when bound together, these two portions form an active transcription factor. The term “portion” when used herein for transcription factor, encompass complete proteins involved in multi protein transcription factors, as well as specific functional protein domains of a complete transcription factor protein.

Therefore in one embodiment of the invention, step a) of the screening method of the invention comprises the following steps:

(1) providing a host cell expressing:
a first fusion polypeptide between (i) a Vangl2 polypeptide as define above and (ii) a first protein portion of transcription factor
a second fusion polypeptide between (i) a p62 polypeptide as defined above and (ii) a second portion of a transcription factor

said transcription factor being active on DNA target regulatory sequence when the first and second protein portion are bound together and

said host cell also containing a nucleic acid comprising (i) a regulatory DNA sequence that may be activated by said active transcription factor and (ii) a DNA report sequence that is operatively linked to said regulatory sequence

(2) bringing said host cell provided at step 1) into contact with a candidate compound to be tested

(3) determining the expression level of said DNA reporter sequence

The expression level of said DNA reporter sequence that is determined at step (3) above is compared with the expression of said DNA reporter sequence when step (2) is omitted. A different expression level of said DNA reporter sequence in the presence of the candidate compound means that the said candidate compound effectively modulates the binding between the Vangl2 polypeptide and the p62 polypeptide and that said candidate compound may be positively selected a step b) of the screening method.

Suitable host cells include, without limitation, prokaryotic cells (such as bacteria) and eukaryotic cells (such as yeast cells, mammalian cells, insect cells, plant cells, etc.). However preferred host cell are yeast cells and more preferably a Saccharomyces cerevisiae cell or a Schizosaccharomyces pombe cell.

Similar systems of two-hybrid assays are well know in the art and therefore can be used to perform the screening method according to the invention (see. Fields et al. 1989; Vasavada et al. 1991; Fearon et al. 1992; Dang et al., 1991, Chien et al. 1991, U.S. Pat. No. 5,283,173, U.S. Pat. No. 5,667,973, U.S. Pat. No. 5,468,614, U.S. Pat. No. 5,525,490 and U.S. Pat. No. 5,637,463). For instance, as described in these documents, the Ga14 activator domain can be used for performing the screening method according to the invention. Ga14 consists of two physically discrete modular domains, one acting as the DNA binding domain, the other one functioning as the transcription-activation domain. The yeast expression system described in the foregoing documents takes advantage of this property. The expression of a Gal1-LacZ reporter gene under the control of a Ga14-activated promoter depends on the reconstitution of Ga14 activity via protein-protein interaction. Colonies containing interacting polypeptides are detected with a chromogenic substrate for β-galactosidase. A compete kit (MATCHMAKER, TM) for identifying protein-protein interactions is commercially available from Clontech.

So in one embodiment, a first Vangl2 polypeptide as above defined is fused to the DNA binding domain of Ga14 and the second p62 polypeptide as above defined is fused to the activation domain of Ga14.

The expression of said detectable marker gene may be assessed by quantifying the amount of the corresponding specific mRNA produced. However, usually the detectable marker gene sequence encodes for detectable protein, so that the expression level of the said detectable marker gene is assessed by quantifying the amount of the corresponding protein produced. Techniques for quantifying the amount of mRNA or protein are well known in the art. For example, the detectable marker gene placed under the control of regulatory sequence may consist of the β-galactosidase as above described.

In another one embodiment, step a) comprises a step of subjecting to a gel migration assay the mixture of the first Vangl2 polypeptide and the second p62 polypeptide as above defined, with or without the candidate compound to be tested and then measuring the binding of the said polypeptides altogether by performing a detection of the complexes formed between said polypeptides. The gel migration assay can be carried out as known by the one skilled in the art.

Therefore in one embodiment of the invention, step a) of the screening method of the invention comprises the following steps:

(1) providing a first Vangl2 polypeptide and a second p62 polypeptide as defined above

(2) bringing into contact the candidate compound to be tested with said polypeptides

(3) performing a gel migration assay a suitable migration substrate with said polypeptides and said candidate compound as obtained at step (2)

(4) detecting and quantifying the complexes formed between said polypeptides on the migration assay as performed at step (3).

The presence or the amount of the complexes formed between the polypeptides are then compared with the results obtained when the assay is performed in the absence of the candidate compound to be tested.

The detection of the complexes formed between the said two polypeptides may be easily performed by staining the migration gel with a suitable dye and then determining the protein bands corresponding to the protein analysed since the complexes formed between the first and the second polypeptides possess a specific apparent molecular weight. Staining of proteins in gels may be done using any well known methods in the art. Suitable gels are well known in the art but it is preferred to use non denaturant gels. In a general manner, western blotting assays are well known in the art and have been widely described (Rybicki et al., 1982; Towbin et al. 1979; Kurien et al. 2006).

In a particular embodiment, the protein bands corresponding to the polypeptides submitted to the gel migration assay can be detected by specific antibodies. It may used both antibodies directed against the Vangl2 polypeptides and antibodies specifically directed against the p62 polypeptides.

In another embodiment, the said two polypeptides are labelled with a detectable antigen as above described. Therefore, the proteins bands can be detected by specific antibodies directed against said detectable antigen. Preferably, the detectable antigen conjugates to the Vangl2 polypeptide is different from the antigen conjugated to the p62 polypeptide. For instance, the first Vangl2 polypeptide can be fused to a GST detectable antigen and the second p62 polypeptide can be fused with the HA antigen. Then the protein complexes formed between the two polypeptides may be quantified and determined with antibodies directed against the GST and HA antigens respectively.

In another embodiment, step a) included the use of an optical biosensor such as described by Edwards et al. (1997) or also by Szabo et al. (1995). This technique allows the detection of interactions between molecules in real time, without the need of labelled molecules. This technique is indeed based on the surface plasmon resonance (SPR) phenomenon. Briefly, a first protein partner is attached to a surface (such as a carboxymethyl dextran matrix). Then the second protein partner is incubated with the previously immobilised first partner, in the presence or absence of the candidate compound to be tested. Then the binding including the binding level, or the absence of binding between said protein partner is detected. For this purpose, a light beam is directed towards the side of the surface area of the substrate that does not contain the sample to be tested and is reflected by said surface. The SPR phenomenon causes a decrease in the intensity of the reflected light with a combination of angle and wavelength. The binding of the first and second protein partner causes a change in the refraction index on the substrate surface, which change is detected as a change in the SPR signal.

In another one embodiment of the invention, the screening method includes the use of affinity chromatography.

Candidate compounds for use in the screening method above can also be selected by any immunoaffinity chromatography technique using any chromatographic substrate onto which (i) the first Vangl2 polypeptide or (ii) the second p62 polypeptide as above defined, has previously been immobilised, according to techniques well known from the one skilled in the art. Briefly, the Vangl2 polypeptide or the p62 polypeptide as above defined may be attached to a column using conventional techniques including chemical coupling to a suitable column matrix such as agarose, Affi Gel®, or other matrices familiar to those of skill in the art. In some embodiment of this method, the affinity column contains chimeric proteins in which the Vangl2 polypeptide or p62 polypeptide as above defined, is fused to glutathion-s-transferase (GST). Then a candidate compound is brought into contact with the chromatographic substrate of the affinity column previously, simultaneously or subsequently to the other polypeptide among the said first and second polypeptide. The after washing, the chromatography substrate is eluted and the collected elution liquid is analysed by detection and/or quantification of the said later applied first or second polypeptide, so as to determine if, and/or to which extent, the candidate compound has modulated the binding between (i) first Vangl2 polypeptide and (ii) the second p62 polypeptide.

In another one embodiment of the screening method according to the invention, the first Vangl2 polypeptide and the second p62 polypeptide as above defined are labelled with a fluorescent molecule or subtrate. Therefore, the potential alteration effect of the candidate compound to be tested on the binding between the first Vangl2 polypeptide and the second p62 polypeptide as above defined is determined by fluorescence quantification.

For example, the first Vangl2 polypeptide and the second p62 polypeptide as above defined may be fused with auto-fluorescent polypeptides, as GFP or YFPs as above described. The first Vangl2 polypeptide and the second p62 polypeptide as above defined may also be labelled with fluorescent molecules that are suitable for performing fluorescence detection and/or quantification for the binding between said polypeptides using fluorescence energy transfer (FRET) assay. The first Vangl2 polypeptide and the second p62 polypeptide as above defined may be directly labelled with fluorescent molecules, by covalent chemical linkage with the fluorescent molecule as GFP or YFP. The first Vangl2 polypeptide and the second p62 polypeptide as above defined may also be indirectly labelled with fluorescent molecules, for example, by non covalent linkage between said polypeptides and said fluorescent molecule. Actually, said first Vangl2 polypeptide and second p62 polypeptide as above defined may be fused with a receptor or ligand and said fluorescent molecule may be fused with the corresponding ligand or receptor, so that the fluorecent molecule can non-covalently bind to said first Vangl2 polypeptide and second p62 polypeptide. A suitable receptor/ligand couple may be the biotin/streptavifin paired member or may be selected among an antigen/antibody paired member. For example, a polypeptide according to the invention may be fused to a poly-histidine tail and the fluorescent molecule may be fused with an antibody directed against the poly-histidine tail.

As already specified, step a) of the screening method according to the invention encompasses determination of the ability of the candidate compound to inhibit the interaction between the Vangl2 polypeptide and the p62 polypeptide as above defined by fluorescence assays using FRET. Thus, in a particular embodiment, the first Vangl2 polypeptide as above defined is labelled with a first fluorophore substance and the second p62 polypeptide is labelled with a second fluorophore substance. The first fluorophore substance may have a wavelength value that is substantially equal to the excitation wavelength value of the second fluorophore, whereby the bind of said first and second polypeptides is detected by measuring the fluorescence signal intensity emitted at the emission wavelength of the second fluorophore substance. Alternatively, the second fluorophore substance may also have an emission wavelength value of the first fluorophore, whereby the binding of said and second polypeptides is detected by measuring the fluorescence signal intensity emitted at the wavelength of the first fluorophore substance.

The fluorophores used may be of various suitable kinds, such as the well-known lanthanide chelates. These chelates have been described as having chemical stability, long-lived fluorescence (greater than 0.1 ms lifetime) after bioconjugation and significant energy-transfer in specificity bioaffinity assay. Document U.S. Pat. No. 5,162,508 discloses bipyridine cryptates. Polycarboxylate chelators with TEKES type photosensitizers (EP0203047A1) and terpyridine type photosensitizers (EP0649020A1) are known. Document WO96/00901 discloses diethylenetriaminepentaacetic acid (DPTA) chelates which used carbostyril as sensitizer. Additional DPT chelates with other sensitizer and other tracer metal are known for diagnostic or imaging uses (e.g., EPO450742A1).

In a preferred embodiment, the fluorescence assay performed at step a) of the screening method consists of a Homogeneous Time Resolved Fluorescence (HTRF) assay, such as described in document WO 00/01663 or U.S. Pat. No. 6,740,756, the entire content of both documents being herein incorporated by reference. HTRF is a TR-FRET based technology that uses the principles of both TRF (time-resolved fluorescence) and FRET. More specifically, the one skilled in the are may use a HTRF assay based on the time-resolved amplified cryptate emission (TRACE) technology as described in Leblanc et al. (2002). The HTRF donor fluorophore is Europium Cryptate, which has the long-lived emissions of lanthanides coupled with the stability of cryptate encapsulation. XL665, a modified allophycocyanin purified from red algae, is the HTRF primary acceptor fluorophore. When these two fluorophores are brought together by a biomolecular interaction, a portion of the energy captured by the Cryptate during excitation is released through fluorescence emission at 620 nm, while the remaining energy is transfered to XL665. This energy is then released by XL665 as specific fluorescence at 665 nm. Light at 665 nm is emitted only through FRET with Europium. Because Europium Cryptate is always present in the assay, light at 620 nm is detected even when the biomolecular interaction does not bring XL665 within close proximity.

Therefore in one embodiment, step a) of the screening method may therefore comprises the steps consisting of:

(1) bringing into contact a pre-assay sample comprising:
a first Vangl2 polypeptide fused to a first antigen,
a second p62 polypeptide fused to a second antigen
a candidate compound to be tested

(2) adding to the said pre assay sample of step (2):
at least one antibody labelled with a European Cryptate which is specifically directed against the first said antigen
at least one antibody labelled with XL665 directed against the second said antigen

(3) illuminating the assay sample of step (2) at the excitation wavelength of the said European Cryptate

(4) detecting and/or quantifying the fluorescence signal emitted at the XL665 emission wavelength

(5) comparing the fluorescence signal obtained at step (4) to the fluorescence obtained wherein pre assay sample of step (1) is prepared in the absence of the candidate compound to be tested.

If at step (5) as above described, the intensity value of the fluorescence signal is different (lower or higher) than the intensity value of the fluorescence signal found when pre assay sample of step (1) is prepared in the absence of the candidate compound to be tested, then the candidate compound may be positively selected at step b) of the screening method.

Antibodies labelled with a European Cryptate or labelled with XL665 can be directed against different antigens of interest including GST, poly-histidine tail, DNP, c-myx, HA antigen and FLAG which include. Such antibodies encompass those which are commercially available from CisBio (Bedfors, Mass., USA), and notably those referred to as 61GSTKLA or 61HISKLB respectively.

The candidate compounds that have been positively selected at the end of any one of the embodiments of the in vitro screening which has been described previously in the present specification may be subjected to further selection steps in view of further assaying its properties on the Vangl2 mediated cellular functions (JNK signaling, cell migration, cell proliferation or tumour growth). For this purpose, the candidate compounds that have been positively selected with the general in vitro screening method as above described may be further selected for their to reduce or inhibit JNK signaling, cell migration, cell proliferation or tumour growth of basal breast cancers.

Thus another aspect of the invention relates to a method for screening a drug for the treatment of breast cancer, wherein said method comprises the steps of: i) screening for compounds that inhibit the interaction between the Vangl2 and the p62 proteins, by performing the in vitro screening method as above described and ii) screening the compounds positively selected at the end of step i) for their ability to inhibit or reduce JNK signaling, cell migration, cell proliferation or tumour growth of basal breast cancers.

In certain preferred embodiments of the screening method above, step ii) of said screening method comprises the following steps:

(1) bringing into contact a cell with a compound that has been positively selected at the end of step i)

(2) determining the capacity of compound to inhibit or reduce JNK signaling, cell migration, cell proliferation or tumour growth of basal breast cancers

(3) comparing the JNK signaling, cell migration, cell proliferation or tumour growth determined at step (2) with the Vangl2 the JNK signaling, cell migration, cell proliferation or tumour growth that are determined when step (1) is performed in the absence of the said positively selected compound, and

(4) positively selecting the compound when the JNK signaling, cell migration, cell proliferation or tumour growth determined at step (2) is lower than the Vangl2 the JNK signaling, cell migration, cell proliferation or tumour growth are determined when step (1) is performed in the absence of the said compound.

Step (1) as above described may be performed by adding an amount of the candidate compound to be tested to the culture medium. Usually, a plurality of culture samples are prepared, so as to add increasing amounts of the candidate compound to be tested in distinct culture samples. Generally, at least one culture sample without candidate compound is also prepared as a negative control for further comparison. Optionally, at least one culture sample with an already known agent that reduces the JNK signaling, cell migration, cell proliferation or tumour growth is also prepared as a positive control for standardisation of the method. Therefore, step (3) may be performed by comparing the percentage of cells wherein the JNK signaling, cell migration, cell proliferation or tumour growth are modulated obtained for the cell cultures incubated with the candidate compound to be tested with the percentage of cells wherein the JNK signaling, cell migration, cell proliferation or tumour growth are modulated obtained for the negative control cell cultures without the said candidate compound. Illustratively, the efficiency of the candidate compound may be assessed by comparing (i) the percentage of cells wherein the JNK signaling, cell migration, cell proliferation or tumour growth are reduced with (ii) the percentage of cells wherein the JNK signaling, cell migration, cell proliferation or tumour growth are reduced measured in the supernatant of the cell cultures that were incubated with the known agent that modulates the JNK signaling, cell migration, cell proliferation or tumour growth. Further illustratively, the efficiency of the candidate compound may be assessed by determining for which amount of the candidate compound added to the cell cultures the percentage of cells wherein the JNK signaling, cell migration, cell proliferation or tumour growth are reduced is close or higher than the percentage of cells wherein the JNK signaling, cell migration, cell proliferation or tumour growth are reduced with the known agent that modulates the JNK signaling, cell migration, cell proliferation or tumour growth.

According to a one embodiment of the invention, the candidate compound of may be selected from the group consisting of peptides, peptidomimetics, small organic molecules, or nucleic acids. For example the candidate compound according to the invention may be selected from a library of compounds previously synthetized, or a library of compounds for which the structure is determined in a database, or from a library of compounds that have been synthetized de novo. In a particular embodiment, the candidate compound may be selected form small organic molecules. As used herein, the term “small organic molecule” refers to a molecule of size comparable to those organic molecules generally sued in pharmaceuticals. The term excludes biological macromolecules (e.g.; proteins, nucleic acids, etc.); preferred small organic molecules range in size up to 2000da, and most preferably up to about 1000 Da.

The present invention relates to a polypeptide having a sequence ranging from the amino acid residue at position 346 to the amino acid residue at position 371 in SEQ ID NO:2 for use in a method for the treatment breast cancer in a patient in need thereof.

The present invention relates to a polypeptide having a sequence ranging from the amino acid residue at position 346 to the amino acid residue at position 388 in SEQ ID NO:2 for use in a method for the treatment breast cancer in a patient in need thereof.

The present invention relates to a polypeptide having a sequence having at least 80% of identity with a sequence ranging from the amino acid residue at position 346 to the amino acid residue at position 371 in SEQ ID NO:2 for use in a method for the treatment breast cancer in a patient in need thereof. Typically, said peptide has at least 80, 81, 82, 83, 84, 85, 86, 87, 88, 89, 90, 91, 92, 93, 94, 95, 96, 97, 98, 99% of identity with the sequence ranging from the amino acid residue at position 346 to the amino acid residue at position 371 in SEQ ID NO:2.

The present invention relates to a polypeptide having a sequence having at least 80% of identity with a sequence ranging from the amino acid residue at position 346 to the amino acid residue at position 388 in SEQ ID NO:2 for use in a method for the treatment breast cancer in a patient in need thereof. Typically, said peptide has at least 80, 81, 82, 83, 84, 85, 86, 87, 88, 89, 90, 91, 92, 93, 94, 95, 96, 97, 98, 99% of identity with the sequence ranging from the amino acid residue at position 346 to the amino acid residue at position 388 in SEQ ID NO:2.

The present invention also relates to a nucleic acid encoding for polypeptide as above described for use in a method for the treatment breast cancer in a patient in need thereof.

The invention will be further illustrated by the following figures and examples. However, these examples and figures should not be interpreted in any way as limiting the scope of the present invention.

Figures:

### EXAMPLE 1

### Identification of a Novel Vangl2/p62 Complex with Tumorigenic Properties in Breast Cancer

Material & Methods

Results

Vangl2 and p62 Form a Strong Endogenous Protein Complex

To find the endogenous protein binding partners of Vangl2 in breast cancer cells, an immunoprecipitation strategy was used to purify endogenous Vangl2 and ascertain its co-immunoprecipitated molecular partners. This was performed in two epithelial breast tumoral cell lines, SKRB7 and SUM149. A previously described monoclonal 2G4 antibody (2G4 mAb) was used which is highly specific to a N-terminal epitope in Vangl2 and unreactive to Vangl1 [29]. Incubation of 2G4 mAb with pre-cleared SKBR7 cell extracts followed by SDS-PAGE separation and visualization by Coomassie blue or silver staining allowed the purification of selected bands that were absent from control samples. These bands were analysed using in-gel digestion, LC-separation and Orbitrap mass spectrometry. Results showed that a 60 kD band present in anti-Vangl2 immunoprecipitates contained endogenous Vangl2, which is absent from the immunoprecipitate carried out with an isotype-matched antibody (HA antibody). Vangl2 was identified with good protein sequence coverage (18%) throughout the entire length of the protein. Furthermore, mass spectrometry analysis allowed the unambiguous identification of p62/sequestosome-1, a cytoplasmic multidomain protein implicated in autophagy, cell signaling and endowed with oncogenic properties, in the 2G4 mAb, but not anti-HA, immunoprecipitates. Like Vangl2, p62 was also identified with good protein sequence coverage (20%) and associated with very good Mascot scores. Peptides used for the identification of Vangl2 and p62 have an ion score above the Mascot Identity Threshold (MIT) and the Mascot Homology Threshold (MHT) values with the significance threshold chosen in our study of p<0.01. The strength of the Vangl2-p62 protein complex was highlighted by its presence in several cell lines (SKBR7, COS-7, SUM149) by immunoprecipitation and western blot suggesting that this complex is biologically important. Conversely, when p62 was immunoprecipitated using a monoclonal p62-specific antibody, Vangl2 was successfully purified from SKBR7 cell extracts. Vangl1 is a close homologue of Vangl2. To evaluate the specificity of the interaction within the Vangl family, we expressed GFP, GFP-Vangl1 and GFP-Vangl2 in COS-7 cells and performed immunoprecipitation with anti-GFP antibody and western blot revealed with anti-p62 antibody. Only GFP-Vangl2 was able to coimmunoprecipitate with p62 further demonstrating the specificity of the Vangl2-p62 interaction.

To evaluate if Vangl2 directly interacts with p62 and identify which region(s) of Vangl2 is (are) required for the interaction, we produced in vitro translated GFP-Vangl2 constructs and performed pulldown assays with a bacterially expressed GST-p62 protein. Full-length Vangl2 (1-521) was able to interact with GST-p62 suggesting a direct interaction. Vangl2 has two cytoplasmic regions, the N-terminal (1-102) and the C-terminal (242-521) regions. Repetition of pulldown assays demonstrated that only the C-terminal Vangl2 region interacts with GST-p62. This region is known to interact with a set of PDZ domain containing proteins through a motif containing serine 464 and through the very last C-terminal TSV motif. Mutation of serine 464 to asparagine (S464N) known to abrogate interaction with the Disheveled PDZ protein and deletion of the TSV motif disrupting interaction with the Scrib PDZ protein, alone or in combination, had no effect on the Vangl2-p62 interaction. Therefore as the (242-472) and (473-521) constructs were able to interact with GST-p62, we concluded that the whole intracellular region of Vangl2 (242-521) is necessary for the interaction with p62.

As the different domains of p62 are highly characterized and implicated in specific protein-protein interactions delegated to different cellular processes, it was important to define which region in p62 was involved in the Vangl2 interaction. This cytoplasmic multi-domain protein contains five distinct domains, i.e. PB1, Zn, TB, LIR and UBA domains. It is well known that p62 forms polymers through its N-terminal PB1 domain. Polymerization can be inhibited by introducing two point mutations (K7A/D69A) within the PB1 domain [35]. We expressed monomeric p62 (p62 K7A/D69A) and evaluated interaction with Vangl2 in transfected COS7 cells. p62 and p62 K7A/D69A were recognized by anti-GFP and anti-p62 antibodies in western blot. Both forms were able to coimmunoprecipitate with Vangl2. Note that less endogenous p62 was recovered in anti-Vangl2 immunoprecipitation when GFP-p62 K7A/D69A was expressed due to lack of p62 polymerization. We next analyzed in details the mode of interaction between Vangl2 and p62 using a panel of EGFP-tagged p62 constructs expressed in COS-7 cells. Immunoprecipitation experiments were performed with 2G4 mAb. The PB1 domain alone (1-122) is not able to coimmunoprecipitate with Vangl2. A longer p62 construct (1-385) that comprises the PB1, Zn, TB and LIR domains could efficiently be recovered with anti-Vangl2 antibody in contrast to p62 (387-440) containing the UBA domain, suggesting that the UBA domain is dispensable for the Vangl2-p62 interaction. Accordingly, a mutant form of p62 containing a killing mutation within the UBA domain (I412A) was able to interact with Vangl2. A deletion of p62 from amino acids 123 to 386 eliminated interaction with Vangl2. We conclude that this region containing the Zn, TB and LIR domains is required for the Vangl2 interaction. We further narrowed down the interaction using mutant forms of GFP-p62 having deletions removing the Zn (123-170) and the TB (170-256) domains, and a region containing the LIR domain (256-370). Only deletion 256-370 was able to abrogate interaction with Vangl2. As deletion 303-349 encompassing the LIR domain did not affect the Vangl2-p62 coimmunoprecipitation, we propose that the p62 (346-388) region is required for the formation of the Vangl2-p62 complex. Of note, this sequence has no known particular feature except a DxxTGE motif (347-352) that represent a binding site for the the Kelch-repeat domain of Kelch-like ECH-associated protein 1 (KEAP1). This motif called the KEAP1 interacting region (KIR) is not involved in Vangl2 interaction as mutations of KIR do not impair interaction. Therefore the region between amino acids residues 346-388 in p62 was potentially important for the interaction with Vangl2 and as yet uncharacterised as binding to other proteins.

Vangl2 and p62 Colocalize in Intracellular Structures:

We next addressed the potential colocalization of Vangl2 and p62 in SUM149 cells by immunofluorescence and confocal analysis. In these cells, Vangl2 and p62 have a punctate cytoplasmic pattern and colocalization between the two proteins was seen puncta in the perinuclear region. Specificity of the Vangl2 signal is attested by lack of staining in cells treated with shVangl2. p62 is present in autophagosomes where it accumulates ubiquitinylated proteins bound to its UBA domain for autophagic degradation. Autophagy is active when the fusion between autophagosomes and lysosomes occur leading to the formation of autophagolysosomes endowed with degradative properties [36]. The fusion process, and therefore autophagy, can be inhibited by Bafilomycin A1, an inhibitor of the vacuolar ATPase that blocks the fusion process by blocking the membrane-bound lysosomal vacuolar ATPase (V-ATPase) to prevent the lysosomal lumen acidification responsible for cathepsin activation [37]. Alternatively, autophagy can be induced by rapamycin, an inhibitor of mTOR [38]. As previously published, treatment of breast cancer and Hela cells with Bafilomycin A1 accumulates p62 in large intracellular structures, presumably autophagosomes [37]. Importantly, p62 was also accumulated and a prominent colocalization between Vangl2 and p62 was observed in these intracellular structures. Rapamycin treatment led in contrast to strong decrease of p62 immunolocalization due to its degradation in autophagolysosomes. Decrease of p62 protein level can also be monitored by western blot. Vangl2 protein levels remained unmodified by rapamycin treatment while another prominent feature of autophagy, i.e. degradation of p62 was readily seen. Strikingly, analysis of the cellular distribution of Vangl2, showed indeed a disappearance of p62 in rapamycin-treated cells, which was accompanied by a redistribution of Vangl2 in smaller intracellular structures that were present in the cytoplasm and at the cell periphery compared to untreated cells. This redistribution of Vangl2 under rapamycin treatment was also seen in SUM149 cells that we also stained for p62 and E-cadherin, a marker of plasma membrane. In western blot experiments, Vangl2 protein levels were not modified by Bafilomycin A1 or by rapamycin treatment. From these data, we conclude that, despite its interaction with p62, Vangl2 is not degraded by autophagy. Redistribution of Vangl2 throughout the cytoplasm following p62 disappearance suggests that p62 anchors Vangl2 within intracellular structures such as autophagosomes.

Vangl2 is Overexpressed in Basal Breast Cancer:

Based on previous studies showing expression of the human Vangl2 gene in breast cancer cells [29], we looked for mRNA expression of this PCP gene in a panel of 35 breast cancer cell lines previously profiled using DNA microarrays [39]. The molecular subtype of cell lines (luminal, mesenchymal, basal) was defined as previously described [40]. Vangl2 is included in the “basal” gene cluster with other genes such as KRT5/6/14 and CRYAB, which is overexpressed in the basal/mesenchymal cell lines such as SKBR7 and SUM149 as compared to luminal cell lines. We next used the 2G4 mAb to characterize Vangl2 in breast cancer samples. This antibody recognizes Vangl2 as three major bands by western blot in breast cancer cells [29] and basal cancer extracts. In comparison, low expression of Vangl2 was detected in a luminal breast cancer sample. In immunohistochemistry assay done on normal breast sample, Vangl2 appears mainly in luminal cells with a membranous and vesicular localization reminiscent to the one found in breast cancer cell lines. We selected sections from breast cancers of luminal and basal subtypes and performed immunohistochemistry. This revealed high expression of Vangl2 in basal but not luminal breast cancer samples. These data demonstrate high expression of Vangl2 in basal breast cancer.

VANGL2 Upregulation is Associated with Poor Prognosis:

We then searched for correlations between VANGL2 mRNA expression and histo-clinical features of tumors in our large data set of 2687 invasive breast cancers, including our series and 14 public microarray data sets.

A total of 767 tumors showed VANGL2 downregulation (29%) as compared to normal breast, 635 upregulation (23%), and 1295 (48%) no deregulation. The human Vangl2 gene is located on chromosome 1q21-q23, a region defined as a cancer susceptibility locus or recombination hot spots in the human genome. Array CGH data (244K Agilent) were available for 208 samples of our series, ˜50% of which showed VANGL2 DNA copy number gain: VANGL2 mRNA upregulation was strongly correlated to gene gain (p=2.9E-06).

Regarding the histo-clinical correlations, deregulated VANGL2 expression was not associated with age and pathological type. By contrast, significant associations existed with the other prognostic pathological features: the up- and the downregulation were associated with higher grade (p=0.002) and larger tumor size (p<0.0001), and the downregulation was associated with axillary lymph node involvement (p=0.023). Interestingly, inverse correlations existed with the molecular parameters: VANGL2 upregulation was associated with more frequent ER-negative status, PR-negative status and ERBB2-negative status, whereas the downregulation was associated with more frequent ER-positive status, PR-positive status and ERBB2-positive status (p<0.0001, p<0.0001, and p=0.023 respectively). Regarding the molecular subtypes, VANGL2-upregulated tumors were more frequently basal (54%), whereas VANGL2-downregulated tumors were more frequently luminal A/B (49%) or ERBB2-overexpressing (24%; p<0.0005).

We then examined the prognostic value of VANGL2 deregulation in the 1208 non-stage IV patients with follow-up available. 492 patients experienced a metastatic relapse after a median time of 25 months from diagnosis, and 716 remained relapse-free with a median follow-up of 84 months. The 5-year MFS was 62% (95CI, 59-65%) for the whole population. In univariate analysis, VANGL2 deregulation was associated with poor MFS (p=0.001): the 5-year MFS was 55% (95CI, 50-62%) in case of upregulation, 60% (95CI, 55-66%) in case of down-regulation, and 66% (95CI, 62-70%) in case of no deregulation. As expected, all other variables tested, except age and pathological type, were associated with MFS: grade, tumor size, axillary lymph node status, and ER, PR and ERBB2 IHC status. In multivariate analysis, VANGL2 upregulation maintained its prognostic value (p=0.020), as well as grade (p=0.045) and axillary lymph node status (p=0.0016), whereas VANGL2 downregulation lost its prognostic value.

Vangl2 is Required for Cell Migration, Cell Proliferation and Tumour Growth:

In order to challenge the role of Vangl2 in tumor growth, we first designed different short hairpin RNA (shRNA) to downregulate Vangl2 protein expression in SUM149, a basal breast cancer cell line, which were targeted to different sequences of the VANGL2 gene. Vangl2 expression is decreased by approximately 90% with shVangl2 (clone 3.6.9 and 3.2.12) compared shLuc controls. Downregulation of PCP components has been shown to impair cell migration. Indeed, shVangl2-SUM149, but not shLuc-SUM149, cells were less responsive to serum used as a chemo-attractant in Boyden chamber assays. This migratory defect was effectively rescued by re-expressing GFP-Vangl2 in shVangl2-SUM149 cells thus demonstrating the specificity of shVangl2. Comparatively overexpression of GFP alone did not hold any rescuing capacity. Behaviour of shLuc-SUM149 and shVangl2-SUM149 cells was next compared in long-term cell proliferation and anchorage-independent experiments using soft agar assays. Cell proliferation and in vitro tumorigenicity of SUM149 cells were decreased when expression of Vangl2 was decreased using shVangl2 clone 3.6.9. This effect was confirmed using a second shVangl2 clone 3.2.12. The tumorigenic potential of Vangl2 was next assessed in vivo by performing subcutaneous xenografts in NOG mice. Orthotopic injections of shLuc-SUM149 and shVangl2-SUM149 cells were performed and tumour growth was periodically measured. A statistically significant decrease of tumour growth was observed in the absence of Vangl2 (p=0.0101). Together, these results show that Vangl2 expression in a basal breast cancer cell is required for cell migration, long-term cell proliferation and tumour growth.

Vangl2-Minimal Binding Region in p62 Acts as a Dominant Negative to Affect JNK Signalling

As JNK signaling is usually activated following PCP activation, we next investigated this pathway in SUM149 cells. As previously shown, down-regulation of Vangl2 leads to a decrease in JNK signaling as shown using a time-dependent FCS stimulation of SUM149 transfected with shVangl2 compared to shLuc. No change in phosphorylation of GSK3 was evidenced in the absence of Vangl2. Conversely, overexpression of Vangl2 in T47D cells that do not express endogenous Vangl2 show increased JNK signaling as well as increased Cdc42 and Rac1 activity as previously described. Knock-down of p62 expression in SUM149 cells phenocopied lack of Vangl2 expression. Since the mapping studies showed that the residues 346-388 of p62 are required for the interaction, and to confirm this hypothesis, we first designed a soluble peptide encompassing this sequence as well as a scramble peptide of identical amino acid composition. Incubation of the p62 (346-388) peptide in SUM149 cell extracts efficiently disrupted the endogenous Vangl2-p62 interaction recovered by immunoprecipitation while no competition was obtained with the scramble peptide. The dose-response showed that concentration of 5 μM was sufficient to disrupt the Vangl2-p62 interaction by 50%. A complete inhibition was obtained with 50 μM of peptide. It was possible to show that a polypeptide reduced in size (346-371) is also able to inhibit endogenous Vangl2-p62 protein complex formation in SUM149 cells. Next, we fused the p62 (346-388) sequence to GFP and expressed the construct in SUM149 cells. As the synthetic p62 peptide, the GFP-p62 (346-388) construct exhibited a dominant-negative effect in co-immunoprecipitation experiments compared to GFP alone. Indeed, this construct was able to specifically coimmunoprecipitate with Vangl2 and furthermore compete with the endogenous Vangl2-p62 interaction as shown by the decreased amount of endogenous p62 associated with Vangl2. In order to assess the contribution of the Vangl2-p62 interaction in JNK signaling, we utilized the dominant negative region of p62 and expressed this as a GFP-tagged protein. Interestingly, GFP-p62 (346-388) was able to decrease JNK signalling as compared to GFP alone mimicking the effect obtained with Vangl2 and p62 downregulation. To finally analyze if this construct was able to functionally exhibit dominant-negative effect in tumorigenesis, soft agar assays and cell migration assays were carried out. Expression of Venus-p62 (346-388) by lentiviral infection led to a decrease of cell migration and to fewer colonies in soft agar assays compared to Venus ctrl. Comparable infection efficiency was shown for GFP-p62 (346-388) and GFP. Taken together, these data demonstrate that the p62 (346-388) sequence exert a dominant-negative effect on the Vangl2-p62 interaction, leading to decreased Vangl2-mediated signaling and tumorigenicity.

### REFERENCES

Throughout this application, various references describe the state of the art to which this invention pertains. The disclosures of these references are hereby incorporated by reference into the present disclosure.

1. Wang, Y. and J. Nathans, Tissue/planar cell polarity in vertebrates: new insights and new questions. Development, 2007. 134(4): p. 647-58.

2. Wu, J. and M. Mlodzik, A quest for the mechanism regulating global planar cell polarity of tissues. Trends Cell Biol, 2009. 19(7): p. 295-305.

3. Adler, P. N., The genetic control of tissue polarity in Drosophila. Bioessays, 1992. 14(11): p. 735-41.

4. Seifert, J. R. and M. Mlodzik, Frizzled/PCP signalling: a conserved mechanism regulating cell polarity and directed motility. Nat Rev Genet, 2007. 8(2): p. 126-38.

5. Kibar, Z., et al., Ltap, a mammalian homolog of Drosophila Strabismus/Van Gogh, is altered in the mouse neural tube mutant Loop-tail. Nat Genet, 2001. 28(3): p. 251-5.

6. Murdoch, J. N., et al., Severe neural tube defects in the loop-tail mouse result from mutation of Lpp1, a novel gene involved in floor plate specification. Hum Mol Genet, 2001. 10(22): p. 2593-601.

7. Fanto, M., et al., Nuclear signaling by Rac and Rho GTPases is required in the establishment of epithelial planar polarity in the Drosophila eye. Curr Biol, 2000. 10(16): p. 979-88.

8. Habas, R., I. B. Dawid, and X. He, Coactivation of Rac and Rho by Wnt/Frizzled signaling is required for vertebrate gastrulation. Genes Dev, 2003. 17(2): p. 295-309.

9. Strutt, D. I., U. Weber, and M. Mlodzik, The role of RhoA in tissue polarity and Frizzled signalling. Nature, 1997. 387(6630): p. 292-5.

10. Fukukawa, C., et al., Activation of the non-canonical Dv1-Rac1-JNK pathway by Frizzled homologue 10 in human synovial sarcoma. Oncogene, 2009. 28(8): p. 1110-20.

11. Zallen, J. A., Planar polarity and tissue morphogenesis. Cell, 2007. 129(6): p. 1051-63.

12. Simons, M. and M. Mlodzik, Planar cell polarity signaling: from fly development to human disease. Annu Rev Genet, 2008. 42: p. 517-40.

13. Kibar, Z., et al., Mutations in VANGL1 associated with neural-tube defects. N Engl J Med, 2007. 356(14): p. 1432-7.

14. Torban, E., et al., Genetic interaction between members of the Vangl family causes neural tube defects in mice. Proc Natl Acad Sci USA, 2008. 105(9): p. 3449-54.

15. Lei, Y. P., et al., VANGL2 mutations in human cranial neural-tube defects. N Engl J Med, 2010. 362(23): p. 2232-5.

16. Montcouquiol, M., et al., Identification of Vangl2 and Scrb1 as planar polarity genes in mammals. Nature, 2003. 423(6936): p. 173-7.

17. Luga, V., et al., Exosomes mediate stromal mobilization of autocrine Wnt-PCP signaling in breast cancer cell migration. Cell, 2012. 151(7): p. 1542-56.

18. Anastas, J. N., et al., A protein complex of SCRIB, NOS1AP and VANGL1 regulates cell polarity and migration, and is associated with breast cancer progression. Oncogene, 2011. 31(32): p. 3696-708.

19. Kaucka, M., et al., The planar cell polarity pathway drives pathogenesis of chronic lymphocytic leukemia by the regulation of B-lymphocyte migration. Cancer Res, 2013.

20. Prebet, T., et al., The cell polarity PTK7 receptor acts as a modulator of the chemotherapeutic response in acute myeloid leukemia and impairs clinical outcome. Blood, 2010. 116(13): p. 2315-23.

21. Huang, L. and S. K. Muthuswamy, Polarity protein alterations in carcinoma: a focus on emerging roles for polarity regulators. Curr Opin Genet Dev, 2010. 20(1): p. 41-50.

22. Audebert, S., et al., Mammalian Scribble forms a tight complex with the betaPlX exchange factor. Curr Biol, 2004. 14(11): p. 987-95.

23. Navarro, C., et al., Junctional recruitment of mammalian Scribble relies on E-cadherin engagement. Oncogene, 2005. 24(27): p. 4330-9.

24. Nola, S., et al., Scrib regulates PAK activity during the cell migration process. Hum Mol Genet, 2008. 17(22): p. 3552-65.

25. Lahuna, O., et al., Thyrotropin receptor trafficking relies on the hScrib-betaPlX-GIT1-ARF6 pathway. EMBO J, 2005. 24(7): p. 1364-74.

26. Cordenonsi, M., et al., The Hippo transducer TAZ confers cancer stem cell-related traits on breast cancer cells. Cell, 2011. 147(4): p. 759-72.

27. Wang, Y., Wnt/Planar cell polarity signaling: a new paradigm for cancer therapy. Mol Cancer Ther, 2009. 8(8): p. 2103-9.

28. Wang, B., et al., Disruption of PCP signaling causes limb morphogenesis and skeletal defects and may underlie Robinow syndrome and brachydactyly type B. Hum Mol Genet, 2010. 20(2): p. 271-85.

29. Belotti, E., et al., Molecular characterisation of endogenous Vangl2/Vangl1 heteromeric protein complexes. PLoS One, 2012. 7(9): p. e46213.

30. Sorlie, T., et al., Gene expression patterns of breast carcinomas distinguish tumor subclasses with clinical implications. Proc Natl Acad Sci USA, 2001. 98(19): p. 10869-74.

31. Sorlie, T., et al., Repeated observation of breast tumor subtypes in independent gene expression data sets. Proc Natl Acad Sci USA, 2003. 100(14): p. 8418-23.

32. Kitamura, H., et al., Cytosolic overexpression of p62 sequestosome 1 in neoplastic prostate tissue. Histopathology, 2006. 48(2): p. 157-61.

33. Thompson, H. G., et al., p62 overexpression in breast tumors and regulation by prostate-derived Ets factor in breast cancer cells. Oncogene, 2003. 22(15): p. 2322-33.

34. Rolland, P., et al., The ubiquitin-binding protein p62 is expressed in breast cancers showing features of aggressive disease. Endocr Relat Cancer, 2007. 14(1): p. 73-80.

35. Lamark, T., et al., Interaction codes within the family of mammalian Phox and Bem1p domain-containing proteins. J Biol Chem, 2003. 278(36): p. 34568-81.

36. Xie, Z. and D. J. Klionsky, Autophagosome formation: core machinery and adaptations. Nat Cell Biol, 2007. 9(10): p. 1102-9.

37. Yamamoto, A., et al., Bafilomycin A1 prevents maturation of autophagic vacuoles by inhibiting fusion between autophagosomes and lysosomes in rat hepatoma cell line, H-4-II-E cells. Cell Struct Funct, 1998. 23(1): p. 33-42.

38. Noda, T. and Y. Ohsumi, Tor, a phosphatidylinositol kinase homologue, controls autophagy in yeast. J Biol Chem, 1998. 273(7): p. 3963-6.

39. Charafe-Jauffret, E., et al., Gene expression profiling of breast cell lines identifies potential new basal markers. Oncogene, 2006. 25(15): p. 2273-84.

40. Ross, D. T. and C. M. Perou, A comparison of gene expression signatures from breast tumors and breast tissue derived cell lines. Dis Markers, 2001. 17(2): p. 99-109.

41. Puissant, A. and P. Auberger, AMPK- and p62/SQSTM1-dependent autophagy mediate Resveratrol-induced cell death in chronic myelogenous leukemia. Autophagy, 2010. 6(5): p. 655-7.

42. Puissant, A., et al., Resveratrol promotes autophagic cell death in chronic myelogenous leukemia cells via JNK-mediated p62/SQSTM1 expression and AMPK activation. Cancer Res, 2010. 70(3): p. 1042-52.

1. A method for predicting the survival of a patient suffering from a breast cancer comprising i) determining the expression level of Vangl2 in a tumor sample obtained from the patient, ii) comparing the expression level determined at step i) with a predetermined reference value and iii) providing a poor prognosis when the expression level determined at step i) is higher than the predetermined reference value.

2. A method for treating a patient suffering from a breast cancer comprising of i) predicting the survival of the patient according to claim 1 and ii) administering to the patient an anti-Vangl2 antibody when it is concluded at step i) that the patient has a poor prognosis.

3. The method according to claim 2 wherein the anti-Vangl2 monoclonal antibody induces antibody dependent cellular cytotoxicity (ADCC) or induces complement dependent cytotoxicity (CDC) against Vangl2-expressing cells or disturbs the expression of Vangl2 at the cell surface so that cell migration, cell proliferation and tumour growth of tumor cells is limited or inhibited.

4. The method according to claim 2 wherein said anti-Vangl2 antibody is selected from the group consisting of a monoclonal antibody, an antigen binding domain, a single domain antibody, a TandAbs dimer, an Fv, an scFv, a dsFv, a ds-scFv, an Fd, a linear antibody, a minibody, a diabody, a bispecific antibody fragment, a bibody, a tribody, a bispecific or trispecific antibody; an sc-diabody; a kappa(lamda) body and a BiTE antibody.

5. The method according to claim 4 wherein the anti-Vangl2 monoclonal antibody is conjugated to a cytotoxic agent or a pro-drug converting enzyme.

6. The method according to claim 4 wherein the anti-Vangl2 antibody is a single domain antibody such as a VHH.

7. The method according to claim 4 wherein the anti-Vangl2 antibody is a bispecific antibody.

8. A method for treating a patient suffering from a breast cancer comprising i) predicting the survival of the patient according to claim 1 and ii) administering to the patient an inhibitor of Vangl2 expression when it is concluded at step i) that the patient has a poor prognosis.

9. A method for treating a patient suffering from a breast cancer comprising i) predicting the survival of the patient according to claim 1 and ii) administering to the patient an mTOR inhibitor when it is concluded at step i) that the patient has a poor prognosis.

10. A method for treating a patient suffering from a breast cancer comprising administering the patient with a therapeutically effective amount of an agent selected from the group consisting of anti-vangl2 antibodies, anti-vangl2 aptamers, inhibitors of Vangl2 expression and mTOR inhibitors.

11. A method for screening a drug for the treatment of breast cancer comprising a) determining the ability of a candidate compound to inhibit the interaction between a Vangl2 polypeptide and a p62 polypeptide and b) positively selecting the candidate compound that inhibits said interaction.

12. A polypeptide having a sequence ranging from an amino acid residue at position 346 to an amino acid residue at position 388 in SEQ ID NO:2 or a sequence having at least 80% identity with the sequence ranging from the amino acid residue at position 346 to the amino acid residue at position 388 in SEQ ID NO:2.

13. The polypeptide of claim 12 having a sequence ranging from the amino acid residue at position 346 to an amino acid residue at position 371 in SEQ ID NO:2 or a sequence having at least 80% of identity with the sequence ranging from the amino acid residue at position 346 to the amino acid residue at position 371 in SEQ ID NO:2.

14. A method for treating breast cancer in a patient in need thereof comprising administering the patient with a therapeutically effective amount of a polypeptide according to claim 12.

15. The method of claim 4, wherein said antigen binding domain is selected from the group consisting of Fab′, Fab, and F(ab′)2.

16. The method of claim 4, wherein said tribody is an scFv-Fab fusion.

17. A method for treating breast cancer in a patient in need thereof comprising administering the patient with a therapeutically effective amount of a polypeptide according to claim 13.
